(19) United States
(12) Patent Application Publication
(10) Pub. No.: US 2019/0351019 A1
(43) Pub. Date: Nov. 21, 2019

(54) STEROID ADMINISTRATION AND IMMUNOTHERAPY

(71) Applicant: Intrexon Corporation, Blacksburg, VA (US)

(72) Inventor: Laurence J.N. Cooper, Houston, TX (US)

(73) Assignee: Intrexon Corporation, Blacksburg, VA (US)

(21) Appl. No.: 16/421,763

(22) Filed: May 24, 2019

Related U.S. Application Data
(63) Continuation of application No. PCT/US2017/064060, filed on Nov. 30, 2017.
(60) Provisional application No. 62/428,474, filed on Nov. 30, 2016.

(51) Int. Cl. A61K 38/19; A61K 38/21; A61K 38/20; A61K 35/17; A61K 31/568; A61K 31/573

(52) U.S. Cl. CPC A61K 38/191; A61K 38/212; A61K 38/2013; A61K 38/2046; A61K 31/573; A61K 38/2086; A61K 35/17; A61K 31/568; A61K 38/208

(57) ABSTRACT

Disclosed herein include methods of inducing expansion of modified effector cells in vivo which comprise administration of a steroid and modified effector cells. Also described herein include methods of inducing expansion of modified T cells (e.g., CAR-T cells or TCR cells) in vivo, which comprise administration of a steroid and modified T cells.

### CROSS-REFERENCE TO RELATED APPLICATION

The present application claims the benefit of U.S. provisional Patent Application No. 62/428,474; filed Nov. 30, 2016, which is hereby incorporated by reference in its entirety.

### BACKGROUND OF THE DISCLOSURE

Cancer is a large and heterogeneous group of diseases, often with treatment response and outcome dependent on the specific type of malignancy. Tumor antigens are useful markers for diagnosis and cancer therapy. In some instances, effector cells can be modified to express exogenous polypeptides that can bind to one or more tumor antigens.

### SUMMARY OF THE DISCLOSURE

Disclosed herein, in certain embodiments, are methods of inducing expansion of modified effector cells in a subject in need thereof. In additional instances, described herein include methods of inducing expansion of modified T cells (e.g., CAR-T cells or TCR-T cells).

Disclosed herein, in certain embodiments, is a method comprising administering to a subject in need thereof: (a) a modified effector cell; and (b) a steroid, such as a corticosteroid, in an amount effective to induce and/or sustain expansion of a population of the modified effector cell(s) in the subject. In some embodiments, the amount does not clinically interfere with engraftment and/or sustained expansion of a population of the modified effector cell(s) in the subject. In some embodiments, the modified effector cell comprises a chimeric receptor. In some embodiments, the modified effector cell is a chimeric antigen receptor (CAR) T cell. In some embodiments, the CAR binds an epitope on CD19. In some embodiments, the CAR binds an epitope on CD33. In some embodiments, the CAR binds an epitope on at least one of BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, Mucins such as MUC-1 or MUC-16, MAGE-A1, h5T4, EGFR, PSMA, TAG-72, EGFRvIII, CD123 and VEGF-R2. In some embodiments, the CAR further comprises at least one costimulatory signaling domain. In some embodiments, the at least one costimulatory signaling domain comprises a signaling domain from CD27, CD28, 4-1BB, ICOS, OX40, CD3-zeta or fragment or combination thereof. In some embodiments, the at least one costimulatory signaling domain comprises a signaling domain from 4-1BB, CD28 or a combination thereof. In some embodiments, the CAR further comprises a CD28 costimulatory signaling domain and CD3-zeta. In some embodiments, the CAR further comprises a CD28 costimulatory signaling domain. In some embodiments, the modified effector cell is a modified natural killer T cell. In some embodiments, the modified natural killer T cell is a modified invariant natural killer T cell. In some embodiments, the modified effector is a T-cell receptor (TCR) engineered T cell. In some embodiments, the modified effector cell is a modified natural killer cell. In some embodiments, the steroid is administered simultaneously with the modified effector cell. In some embodiments, the steroid is a corticosteroid. In some embodiments, the steroid comprises fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol. In some embodiments, the steroid is administered sequentially with the modified effector cell. In some embodiments, the steroid is administered to the subject prior to administering the modified effector cell. In some embodiments, the steroid is administered to the subject after administering the modified effector cell. In some embodiments, the steroid is administered at least 0.5, 1, 2, 3, 4, 5, 6, 8, 10, 12, 15, 18, 20, or 24 hours prior to administration of the modified effector cell. In some embodiments, the steroid is administered at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 days prior to administration of the modified effector cell. In some embodiments, the steroid is administered at least 0.5, 1, 2, 3, 4, 5, 6, 8, 10, 12, 15, 18, 20, 24, 36, 48, 60, 72, 84, 96, 108, or 120 hours after administration of the modified effector cell. In some embodiments, the steroid is administered at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified effector cell. In some embodiments, the steroid is administered continuously for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the steroid is administered at predetermined time intervals for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the steroid is administered every 10 days. In some embodiments, the amount of steroid is administered in 1 dose, 2 doses, 3 doses, 4 doses, 5 doses or more. In some embodiments, a first amount of steroid is administered in 1 dose, 2 doses, 3 doses, 4 doses, 5 doses or more for a first predetermined amount of time; and a second amount of steroid is administered in 1 dose, 2 doses, 3 doses, 4 doses, 5 doses or more for a second predetermined amount of time. In some embodiments, the first predetermined amount of time is 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 days. In some embodiments, the second predetermined amount of time is 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 days. In some embodiments, a first amount of steroid is administered to the subject for a first period of time, and a second amount of steroid is administered to the subject for a second period of time. In some embodiments, the first amount of steroid is administered to the subject in 1 dose, 2 doses, 3 doses, 4 doses, 5 doses or more for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is an amount sufficient for treating a condition. In some embodiments, the condition is graft versus host disease (GVHD). In some embodiments, the condition is cytokine release syndrome (CRS). In some embodiments, upon recovery or improvement of the condition, said second amount of steroid is administered to the subject. In some embodiments, the second amount of steroid is administered to the subject in 1 dose, 2 doses, 3 doses, 4 doses, 5 doses or more for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered to the subject simultaneously with the modified effector cell. In some embodiments, the first amount of steroid is administered to the subject after administering the modified effector cell. In some embodiments, the first amount of steroid is administered to the subject prior to administering the modified effector cell. In some embodiments, the first amount of steroid is administered to the subject at least 1, 2, 3, 4, 5, 6, 7, 8, 9, 10, 12, 13, 14, or 15 days after administration of the modified effector cell. In some embodiments, the first amount of steroid is administered to the subject at least 15 or more days after administration of the modified effector cell. In some embodiments, the first amount of steroid is administered to the subject continuously or intermittently for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered to the subject at predetermined time intervals for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the second amount of steroid is administered to the subject at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified effector cell and after administration of the first amount of steroid has been completed. In some embodiments, the second amount of steroid is administered to the subject at least 49 or more days after administration of the modified effector cell and after administration of the first amount of steroid has been completed. In some embodiments, the first amount of steroid is 1 mg/kg. In some embodiments, the second amount of steroid is about 0.9 mg/kg to about 0.5 mg/kg. In some embodiments, the second amount of steroid is 0.5 mg/kg. In some embodiments, the second amount of steroid is administered in sequential doses. In some embodiments, each sequential dose is reduced by 0.1 mg/kg. In some embodiments, the method further comprises administering a cytokine. In some embodiments, the cytokine comprises an interferon, an interleukin, a chemokine, a colony-stimulating factor or a tumor necrosis factor. In some embodiments, the cytokine is co-expressed with the modified effector cell. In some embodiments, the cytokine comprises IL-2, IL-7, IL-12, IL-15, IL-21, IFNγ or TNF-α. In some embodiments, the cytokine comprises membrane-bound IL-15 (mbIL-15). In some embodiments, the cytokine is administered simultaneously with the steroid or the modified effector cell. In some embodiments, the cytokine is administered simultaneously with at least one of the steroid and the modified effector cell. In some embodiments, the cytokine and modified effector cells are administered simultaneously or sequentially, and the steroid is administered prior to administration of the cytokine and modified effector cells or after the administration of the cytokine and modified effector cells but prior to any symptoms associated with a cytokine storm. In some embodiments, the cytokine is administered sequentially with the steroid and/or the modified effector cell. In some embodiments, the steroid is administered upon manifestation of at least one symptom of cytokine-associated toxicity selected from cytokine release syndrome (CRS), encephalopathy and B-cell aplasia. In some embodiments, the steroid is administered before manifestation of CRS, encephalopathy, and B-cell aplasia. In some embodiments, the cytokine-associated toxicity induces cytokine storm. In some embodiments, the steroid is administered in an amount that is less than an amount of steroid needed for a reduction in at least one symptom of cytokine-associated toxicity selected from cytokine release syndrome (CRS), encephalopathy and B-cell aplasia. In some embodiments, the steroid is administered in an amount of about 1 mg/kg. In some embodiments, the steroid is administered in an amount of about 0.5 mg/kg. In some embodiments, the steroid and/or the modified effector cell is formulated for at least one of parenteral, subcutaneous, sublingual, intranasal and oral administration. In some embodiments, an amount of the modified effector cell is administered to the subject. In some embodiments, the amount is about 10⁵ to about 10⁹ modified effector cells/kg. In some embodiments, the amount is about 10⁵ to about 10⁶ modified effector cells/kg. In some embodiments, the amount is about 10⁶ to about 10⁷ modified effector cells/kg. In some embodiments, the amount is about 10⁷ to about 10⁸ modified effector cells/kg. In some embodiments, the amount is about 10⁸ to about 10⁹ modified effector cells/kg. In some embodiments, the amount is about 10⁵ modified effector cells/kg. In some embodiments, the subject has a cancer. In some embodiments, the cancer is a solid tumor. In some embodiments, the solid tumor comprises breast cancer, cervical cancer, colorectal cancer, kidney cancer, liver cancer, lung cancer, ovarian cancer, pancreatic cancer, prostate cancer or urothelial cancer. In some embodiments, the cancer is a hematologic malignancy. In some embodiments, the hematologic malignancy comprises leukemia, a lymphoma or a myeloma. In some embodiments, the hematologic malignancy comprises a B-cell malignancy. In some embodiments, the B-cell malignancy comprises chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic malignancy comprises a myeloid leukemia. In some embodiments, the hematologic malignancy comprises acute myeloid leukemia (AML) or chronic myeloid leukemia (CML). In some embodiments, the cancer is a metastatic cancer. In some embodiments, AML is a relapsed AML. In some embodiments, AML is a refractory AML. In some embodiments, the cancer is a relapsed or refractory cancer. In some embodiments the recipient has overt malignant disease. In some embodiments the recipient has minimal residual malignant disease. In some embodiments, the subject is a human. In some instances, the subject is a human aged 18 or older. In other instances, the subject is a human aged 17 or younger. In some instances, the subject is a human infant child or adolescent.

Disclosed herein, in certain embodiments, is a method comprising administering to a subject: (a) a CAR-T cell which binds an epitope on CD19; and (b) a steroid, wherein the steroid is concurrently or sequentially administered with the CAR-T cell, and the steroid is administered to the subject in an amount effective to induce and/or sustain expansion of a population of the CAR-T cell in the subject. In some embodiments, the amount does not adversely impact engraft and/or sustained expansion of a population of the CAR-T cell in the subject. In some embodiments, the steroid is administered prior to manifestation of at least one symptom of a cytokine storm. In some cases, the steroid is administered at an amount lower than an amount sufficient to treat at least one symptom of a cytokine storm. In some embodiments, the CAR-T cell further comprises at least one costimulatory signaling domain. In some embodiments, the at least one costimulatory signaling domain comprises a signaling domain from CD27, CD28, 4-1BB, ICOS, OX40, CD3-zeta or fragment or combination thereof. In some embodiments, the at least one costimulatory signaling domain comprises a signaling domain from 4-1BB, CD28 or a combination thereof. In some embodiments, the CAR-T cell further comprises a CD28 costimulatory signaling domain and CD3-zeta. In some embodiments, the CAR further comprises a CD28 costimulatory signaling domain. In some embodiments, the modified effector cell is a modified natural killer T cell. In some embodiments, the modified natural killer T cell is a modified invariant natural killer T cell. In some embodiments, the modified effector is a T-cell receptor (TCR) engineered T cell. In some embodiments, the modified effector cell is a modified natural killer cell. In some embodiments, the steroid is administered simultaneously with the modified effector cell. In some embodiments, the steroid is administered sequentially with the modified effector cell. In some embodiments, the steroid is administered to the subject prior to administering the modified effector cell. In some embodiments, the steroid is administered to the subject after administering the modified effector cell. In some embodiments, the steroid is administered at least 0.5, 1, 2, 3, 4, 5, 6, 8, 10, 12, 15, 18, 20, or 24 hours prior to administration of the modified effector cell. In some embodiments, the steroid is administered at least 2, 3, 4, 5, 6, 7, 8, 9, 10, 12, 14, 15, 28 or 30 days prior to administration of the modified effector cell. In some embodiments, the steroid is administered at least 0.5, 1, 2, 3, 4, 5, 6, 8, 10, 12, 15, 18, 20, 24, 36, 48, 60, 72, 84, 96, 108, or 120 hours after administration of the modified effector cell. In some embodiments, the steroid is administered at least 2, 3, 4, 5, 6, 7, 8, 9, 10, 12, 14, 15, 28, 30, 45, 60, 90 or more days after administration of the modified effector cell. In some embodiments, the steroid is administered continuously for 1, 2, 3, 4, 5, 6, 7, 8, 9, 10, 12, 15, 20, 25, 30, 45, 60, 90 or more days. In some embodiments, the steroid is administered at predetermined time intervals for 1, 2, 3, 4, 5, 6, 7, 8, 9, 10, 12, 15, 20, 25, 30, 45, 60, 90 or more days. In some embodiments, the amount of steroid is administered in 1 dose, 2 doses, 3 doses, 4 doses, 5 doses or more. In some embodiments, a first amount of steroid is administered in 1 dose, 2 doses, 3 doses, 4 doses, 5 doses or more for treating a condition; and upon recovery or improvement of the condition, a second amount of steroid is administered in 1 dose, 2 doses, 3 doses, 4 doses, 5 doses or more. In some embodiments, the first amount of steroid is 1 mg/kg. In some embodiments, the second amount of steroid is 0.5 mg/kg. In some embodiments, the steroid comprises fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol. In some embodiments, the method further comprises administering a cytokine. In some embodiments, the cytokine comprises an interferon, an interleukin, a chemokine, a colony-stimulating factor or a tumor necrosis factor. In some embodiments, the cytokine is co-expressed with the modified effector cell. In some embodiments, the cytokine comprises IL-2, IL-7, IL-12, IL-15, IL-21, IFNγ or TNF-α. In some embodiments, the cytokine comprises mbIL-15. In some embodiments, the cytokine is administered simultaneously with the steroid or the modified effector cell. In some embodiments, the cytokine is administered simultaneously with the steroid and the modified effector cell. In some embodiments, the cytokine is administered sequentially with the steroid and/or the modified effector cell. In some embodiments, the steroid is administered upon manifestation of at least one symptom of cytokine-associated toxicity selected from cytokine release syndrome (CRS), encephalopathy and B-cell aplasia. In some embodiments, the cytokine-associated toxicity induces cytokine storm. In some embodiments, the steroid is administered in an amount that is less than an amount of steroid needed for a reduction in at least one symptom of cytokine-associated toxicity selected from cytokine release syndrome (CRS), encephalopathy and B-cell aplasia. In some embodiments, the steroid and/or the modified effector cell is formulated for at least one of parenteral, subcutaneous, sublingual, intranasal and oral administration. In some embodiments, the subject has a cancer. In some embodiments, the cancer is a solid tumor. In some embodiments, the solid tumor comprises breast cancer, cervical cancer, colorectal cancer, kidney cancer, liver cancer, lung cancer, ovarian cancer, pancreatic cancer, prostate cancer or urothelial cancer. In some embodiments, the cancer is a hematologic malignancy. In some embodiments, the hematologic malignancy comprises a leukemia, a lymphoma or a myeloma. In some embodiments, the hematologic malignancy comprises a B-cell malignancy. In some embodiments, the B-cell malignancy comprises chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic malignancy comprises a myeloid leukemia. In some embodiments, the hematologic malignancy comprises acute myeloid leukemia (AML) or chronic myeloid leukemia (CML). In some embodiments, the cancer is a metastatic cancer. In some embodiments, the cancer is a relapsed or refractory cancer. In some embodiments, the subject is a human. In some instances, the subject is a human aged 18 or older. In other instances, the subject is a human aged 17 or younger. In some instances, the subject is a human infant child or adolescent.

Disclosed herein, in certain embodiments, is a method of inducing T cell engraftment and/or expansion in a subject in need thereof, comprising: (a) contacting a T cell ex vivo with a vector encoding a chimeric antigen receptor to generate a modified T cell; (b) administering to the subject an amount of the modified T cell; and (c) administering to the subject a steroid in an amount effective to induce and/or sustain expansion of a population of the modified T cells in the subject. In some embodiments, the amount does not clinically interfere with engraftment and/or sustained expansion of a population of the modified effector cell(s) in the subject. In some embodiments, the vector is a lentivirus vector, a retroviral vector, a Sleeping Beauty transposon or a non-viral vector. In some embodiments, the vector is a Sleeping Beauty transposon. In some embodiments, the modified T cell is a chimeric antigen receptor (CAR) T cell. In some embodiments, the modified T cell is an engineered T-cell receptor (TCR) T cell. In some embodiments, the T cell is modified at a point-of-care site and administered to the subject without undergoing propagation and activation. In some embodiments, the modified T cells are stimulated for at least 0, 2, 4, 6, 8, 10, 12, 14, 16, 18, 20, 22, 24, 26, 28, 30 or more days. In some embodiments, the modified T cells are stimulated for at least 5, 10, 15, 20, 25, 30, 35, 40, 45, 50, 55, 60 or more days. In some embodiments, the modified T cells are stimulated for at least 7, 14, 21, 28, 35, 42, 49, 56, 63 or more days. In some embodiments, the chimeric antigen receptor bind to an epitope on at least one of CD19, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MAGE-A1, h5T4, PSMA, TAG-72, EGFRvIII, CD123 and VEGF-R2. In some embodiments, the CAR-T cell further comprises at least one costimulatory signaling domain. In some embodiments, the at least one costimulatory signaling domain comprises a signaling domain from CD27, CD28, 4-1BB, ICOS, OX40, CD3-zeta or fragment or combination thereof. In some embodiments, the at least one costimulatory signaling domain comprises a signaling domain from 4-1BB, CD28 or a combination thereof. In some embodiments, the modified T cell further comprises a CD28 costimulatory signaling domain and CD3-zeta. In some embodiments, the modified T cell further comprises costimulatory signaling domains CD28. In some embodiments, the modified effector cell is a modified natural killer T cell. In some embodiments, the modified natural killer T cell is a modified invariant natural killer T cell. In some embodiments, the steroid is administered simultaneously with the modified T cell. In some embodiments, the steroid is administered sequentially with the modified T cell. In some embodiments, the steroid is administered to the subject prior to administering the modified T cell. In some embodiments, the steroid is administered to the subject after administering the modified T cell. In some embodiments, the steroid is administered at least 0.5, 1, 2, 3, 4, 5, 6, 8, 10, 12, 15, 18, 20, or 24 hours prior to administration of the modified T cell. In some embodiments, the steroid is administered at least 2, 3, 4, 5, 6, 7, 8, 9, 10, 12, 14, 15, 28 or 30 days prior to administration of the modified effector cell. In some embodiments, the steroid is administered at least about 0.5, 1, 2, 3, 4, 5, 6, 8, 10, 12, 15, 18, 20, 24, 36, 48, 72, 80, 90, 100, 108, 120, 130, 140, 150 or 160 hours after administration of the modified T cell. In some embodiments, the steroid is administered at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified effector cell. In some embodiments, the steroid is administered continuously for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the steroid is administered at predetermined intervals for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days optionally in combination with the modified T cell. In some embodiments, the steroid is administered every 10 days. In some embodiments, the amount of steroid is administered in 1 dose, 2 doses, 3 doses, 4 doses, 5 doses or more. In some embodiments, a first amount of steroid is administered to the subject. In some embodiments, the first amount of steroid is administered to the subject in 1 dose, 2 doses, 3 doses, 4 doses, 5 doses or more. In some embodiments, the first amount of steroid is administered to the subject for treating a condition. In some embodiments, the condition is graft versus host disease (GVHD). In some embodiments, the condition is cytokine release syndrome (CRS). In some embodiments, upon recovery or improvement of the condition, a second amount of steroid is administered to the subject. In some embodiments, the second amount of steroid is administered in 1 dose, 2 doses, 3 doses, 4 doses, 5 doses or more. In some embodiments, the first amount of steroid is administered to the subject simultaneously with the modified T cell. In some embodiments, the first amount of steroid is administered to the subject after administering the modified T cell. In some embodiments, the first amount of steroid is administered to the subject prior to administering the modified T cell. In some embodiments, the first amount of steroid is administered to the subject at least 2, 3, 4, 5, 6, 7, 8, 9, 10, 12, 14, 15, 28, 30, 45, 60, 90 or more days after administration of the modified T cell. In some embodiments, the first amount of steroid is administered to the subject at least 15 or more days after administration of the modified T cell. In some embodiments, the first amount of steroid is administered to the subject continuously for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered to the subject at predetermined time intervals for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the second amount of steroid is administered to the subject at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified T cell and after administration of the first amount of steroid has been completed. In some embodiments, the second amount of steroid is administered to the subject at least 49 or more days after administration of the modified T cell and after administration of the first amount of steroid has been completed. In some embodiments, the second amount of steroid is administered to the subject continuously for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the second amount of steroid is administered to the subject at predetermined time intervals for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is 1 mg/kg. In some embodiments, the second amount of steroid is about 0.9 mg/kg to about 0.5 mg/kg. In some embodiments, the second amount of steroid is 0.5 mg/kg. In some embodiments, the second amount of steroid is administered in sequential doses. In some embodiments, each sequential dose is reduced by 0.1 mg/kg. In some embodiments, the steroid comprises fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol. In some embodiments, the steroid is administered in an amount that is less than an amount of steroid needed for a reduction in at least one symptom of cytokine-associated toxicity. In some embodiments, the steroid is administered in an amount sufficient for a reduction in at least one symptom of cytokine-associated toxicity. In some embodiments, said at least one symptom of cytokine-associated toxicity is selected from cytokine release syndrome (CRS), encephalopathy and B-cell aplasia. In some embodiments, the steroid is administered in an amount of about 1 mg/kg. In some embodiments, the steroid is administered in an amount of about 0.5 mg/kg. In some embodiments, the amount of the modified T cell is about 10⁵ to about 10⁹ modified T cells/kg. In some embodiments, the amount of the modified T cell is about 10⁵ to about 10⁶ modified T cells/kg. In some embodiments, the amount of the modified T cell is about 10⁶ to about 10⁷ modified T cells/kg. In some embodiments, the amount of the modified T cell is about 10⁷ to about 10⁸ modified T cells/kg. In some embodiments, the amount of the modified T cell is about 10⁸ to about 10⁹ modified T cells/kg. In some embodiments, the amount of the modified T cell is about 10⁵ modified T cells/kg. In some embodiments, the method further comprises administering a cytokine. In some embodiments, the cytokine comprises an interferon, an interleukin, a chemokine, a colony-stimulating factor or a tumor necrosis factor. In some embodiments, the cytokine is co-expressed with the modified T cell. In some embodiments, the cytokine comprises IL-2, IL-7, IL-12, IL-15, IL-21, IFNγ or TNF-α. In some embodiments, the cytokine comprises mbIL-15. In some embodiments, the cytokine is administered simultaneously with the steroid or the CAR-T modified T cell. In some embodiments, the cytokine is administered simultaneously with the steroid and the modified T cell. In some embodiments, the cytokine is administered sequentially with the steroid and/or the modified T cell. In some embodiments, the steroid and/or the modified T cell is formulated for parenteral administration. In some embodiments, the steroid and/or the modified T cell is formulated for oral administration. In some embodiments, the subject is a human. In some instances, the subject is a human aged 18 or older. In other instances, the subject is a human aged 17 or younger. In some instances, the subject is a human infant child or adolescent.

Disclosed herein, in certain embodiments, is a method of inducing T cell expansion in a subject in need thereof, comprising: (a) contacting a T cell ex vivo with a vector comprising a nucleic acid encoding a chimeric antigen receptor that recognizes an epitope on CD19 to generate a CD19 CAR-T cell; and (b) administering to the subject a combination of a population of CD19-specific T cells and an amount of steroid, wherein the amount of steroid is effective to induce and/or sustain expansion of the CD19-specific T cells in the subject. In some embodiments, the amount does not clinically interfere with engraftment and/or sustained expansion of a population of the modified effector cell(s) in the subject. In some embodiments, the vector is a lentivirus vector, a retroviral vector, a Sleeping Beauty transposon or a non-viral vector. In some embodiments, the vector is a Sleeping Beauty transposon. In some embodiments, the CAR-T cells are stimulated for at least 0, 2, 4, 6, 8, 10, 12, 14, 16, 18, 20, 22, 24, 26, 28, 30 or more days. In some embodiments, the CAR-T cells are stimulated for at least 5, 10, 15, 20, 25, 30, 35, 40, 45, 50, 55, 60 or more days. In some embodiments, the CAR-T cells are stimulated for at least 7, 14, 21, 28, 35, 42, 49, 56, 63 or more days. In some embodiments, the CAR-T cell further comprises at least one costimulatory signaling domain. In some embodiments, the at least one costimulatory signaling domain comprises a signaling domain from CD27, CD28, 4-1BB, ICOS, OX40, CD3-zeta or fragment or combination thereof. In some embodiments, the at least one costimulatory signaling domain comprises a signaling domain from 4-1BB, CD28 or a combination thereof. In some embodiments, the CAR-T cell further comprises a CD28 costimulatory signaling domain and CD3-zeta. In some embodiments, the CAR-T further comprises a CD28 costimulatory signaling domain. In some embodiments, the modified effector cell is a modified natural killer T cell. In some embodiments, the modified natural killer T cell is a modified invariant natural killer T cell. In some embodiments, the steroid is administered simultaneously with the CAR-T cell. In some embodiments, the steroid is administered sequentially with the CAR-T cell. In some embodiments, the steroid is administered to the subject prior to administering the CAR-T cell. In some embodiments, the steroid is administered to the subject after administering the CAR-T cell. In some embodiments, the steroid is administered at least 0.5, 1, 2, 3, 4, 5, 6, 8, 10, 12, 15, 18, 20, or 24 hours prior to administration of the CAR-T cell. In some embodiments, the steroid is administered at least 2, 3, 4, 5, 6, 7, 8, 9, 10, 12, 14, 15, 28 or 30 days prior to administration of the modified effector cell. In some embodiments, the steroid is administered at least about 0.5, 1, 2, 3, 4, 5, 6, 8, 10, 12, 15, 18, 20, 24, 36, 48, 72, 80, 90, 100, 108, 120, 130, 140, 150 or 160 hours after administration of the CAR-T cell. In some embodiments, the steroid is administered at least 2, 3, 4, 5, 6, 7, 8, 9, 10, 12, 14, 15, 28, 30, 45, 60, 90 or more days after administration of the modified effector cell. In some embodiments, the steroid is administered continuously for 1, 2, 3, 4, 5, 6, 7, 8, 9, 10, 12, 15, 20, 25, 30, 45, 60, 90 or more days. In some embodiments, the steroid is administered at predetermined intervals for 1, 2, 3, 4, 5, 6, 7, 8, 9, 10, 12, 15, 20, 25, 30, 45, 60, 90 or more days optionally in combination with the modified CAR-T cell. In some embodiments, the amount of steroid is administered in 1 dose, 2 doses, 3 doses, 4 doses, 5 doses or more. In some embodiments, a first amount of steroid is administered in 1 dose, 2 doses, 3 doses, 4 doses, 5 doses or more for treating a condition; and upon recovery or improvement of the condition, a second amount of steroid is administered in 1 dose, 2 doses, 3 doses, 4 doses, 5 doses or more. In some embodiments, the first amount of steroid is 1 mg/kg. In some embodiments, the second amount of steroid is 0.5 mg/kg. In some embodiments, the steroid comprises fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol. In some embodiments, the steroid is administered in an amount that is less than an amount of steroid needed for a reduction in at least one symptom of cytokine-associated toxicity. In some embodiments, the steroid is administered in an amount sufficient for a reduction in at least one symptom of cytokine-associated toxicity. In some embodiments, said at least one symptom of cytokine-associated toxicity is selected from cytokine release syndrome (CRS), encephalopathy and B-cell aplasia. In some embodiments, the method further comprises administering a cytokine. In some embodiments, the cytokine comprises an interferon, an interleukin, a chemokine, a colony-stimulating factor or a tumor necrosis factor. In some embodiments, the cytokine is co-expressed with the CAR-T cell. In some embodiments, the cytokine comprises IL-2, IL-7, IL-12, IL-15, IL-21, IFNγ or TNF-α. In some embodiments, the cytokine comprises mbIL-15. In some embodiments, the cytokine is administered simultaneously with the steroid or the CAR-T cell. In some embodiments, the cytokine is administered simultaneously with the steroid and the CAR-T cell. In some embodiments, the cytokine is administered sequentially with the steroid and/or the CAR-T cell. In some embodiments, the steroid and/or the CAR-T cell is formulated for parenteral administration. In some embodiments, the steroid and/or the CAR-T cell is formulated for oral administration. In some embodiments, the chimeric receptor is a chimeric antigen receptor (CAR). In some embodiments, the chimeric receptor is an antigen-specific engineered T cell receptor. In some embodiments, the T cell is further contacted with a vector encoding a cytokine. In some embodiments, the cytokine comprises mbIL-15 or a fusion protein thereof.

Disclosed herein, in certain embodiments, is a method of inducing T cell expansion in vivo, comprising: contacting a population of modified T cells in vivo with a first amount of steroid sufficient to induce and/or sustain expansion of the population of the modified T cells, wherein said modified T cells comprise at least one chimeric receptor expressed on the cell surface. In some embodiments, the method further comprises administering the population of modified T cells to a subject in need thereof. In some embodiments, the population of modified T cells is about 10⁵ to about 10⁹ modified T cells/kg. In some embodiments, the population of modified T cells is about 10⁵ to about 10⁶ modified T cells/kg. In some embodiments, the population of modified T cells is about 10⁶ to about 10⁷ modified T cells/kg. In some embodiments, the population of modified T cells is about 10⁷ to about 10⁸ modified T cells/kg. In some embodiments, the population of modified T cells is about 10⁸ to about 10⁹ modified T cells/kg. In some embodiments, the population of modified T cells is about 10⁵ modified T cells/kg. In some embodiments, the first amount of steroid is administered at least 0.5, 1, 2, 3, 4, 5, 6, 8, 10, 12, 15, 18, 20, 24, 36, 48, 60, 72, 84, 96, 108, or 120 hours after administration of the modified T cell. In some embodiments, the first amount of steroid is administered at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified T cell. In some embodiments, the method further comprises contacting a T cells ex vivo with a vector encoding the chimeric receptor to generate the modified T cell, wherein said contacting is performed prior to contacting said T cells in vivo with said first amount of steroid. In some embodiments, the vector is a lentivirus vector, a retroviral vector or a Sleeping Beauty transposon vector. In some embodiments, the vector is a Sleeping Beauty transposon. In some embodiments, the T cell is modified at a point-of-care site and administered to the subject without undergoing propagation and activation. In some embodiments, said at least one chimeric receptor is a chimeric antigen receptor (CAR). In some embodiments, the at least one chimeric receptor is an antigen-specific engineered T cell receptor. In some embodiments, the modified T cell is stimulated for at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. In some embodiments, the modified T cell is stimulated for at least 5, 10, 15, 20, 25, 30, 35, 40, 45, 50, 55, 60 or more days. In some embodiments, the modified T cell is stimulated for at least 7, 14, 21, 28, 35, 42, 49, 56, 63 or more days. In some embodiments, the at least one chimeric receptor binds to an epitope on at least one of CD19, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MAGE-A1, h5T4, PSMA, TAG-72, EGFRvIII, CD123 and VEGF-R2. In some embodiments, the modified T cell further comprises at least one costimulatory signaling domain. In some embodiments, the at least one costimulatory signaling domain comprises a signaling domain from CD27, CD28, 4-1BB, ICOS, OX40 or fragment or combination thereof. In some embodiments, the at least one costimulatory signaling domain comprises a signaling domain from 4-1BB, CD28 or a combination thereof. In some embodiments, the modified T cell further comprises a CD28 costimulatory signaling domain and CD3-zeta. In some embodiments, the modified T cell further comprises a CD28 costimulatory signaling domain. In some embodiments, the first amount of steroid is administered continuously for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered at predetermined time intervals for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered every 10 days. In some embodiments, the first amount of steroid is administered in 1 dose, 2 doses, 3 doses, 4 doses, 5 doses or more. In some embodiments, the first amount of steroid is less than an amount of steroid needed for a reduction in at least one symptom of cytokine-associated toxicity. In some embodiments, the first amount of steroid is about 1 mg/kg. In some embodiments, the population of modified T cells in vivo is further contacted with a second amount of steroid subsequent to contacting with said first amount of steroid. In some embodiments, said second amount of steroid is provided at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after contacting with said first amount of steroid. In some embodiments, the contact with second amount of steroid is at least 30 or more days after said contact with said first amount of steroid. In some embodiments, the second amount of steroid is lower than the first amount of steroid. In some embodiments, the second amount of steroid is about 0.9 mg/kg to about 0.5 mg/kg. In some embodiments, the second amount of steroid is about 0.5 mg/kg. In some embodiments, the second amount of steroid is administered in sequential doses. In some embodiments, each sequential dose is reduced by 0.1 mg/kg. In some embodiments, the second amount of steroid is administered continuously for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the additional amount of steroid is administered at predetermined time intervals for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the additional amount of steroid is administered every 10 days. In some embodiments, the additional amount of steroid is administered in 1 dose, 2 doses, 3 doses, 4 doses, 5 doses or more. In some embodiments, the steroid comprises fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol.

Disclosed herein, in certain embodiments, is a kit comprising a modified effector cell and optionally a steroid for use with a method described herein.

Disclosed herein, in certain embodiments, is a system for inducing modified effector cell population expansion in vivo, comprising a population of modified T cells, and an amount of at least one steroid, wherein contacting a population of said modified effector cell in vivo with an effective amount of said at least one steroid, results in an expansion of the population of said modified effector cell. In some embodiments, the amount is effective to induce and/or sustain expansion. In some embodiments, said amount of said at least one steroid is less than an amount of steroid needed for a reduction in at least one symptom of cytokine-associated toxicity. In some embodiments, the modified effector cell is a chimeric antigen receptor (CAR) T cell. In some embodiments, the at least one steroid comprises at least one of fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone and stanozolol.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a PET-CT scan with ¹⁸F-fluorodeoxyglucose (¹⁸F-FDG) prior to the administration of the CAR-T cells.

FIG. 2 shows a PET-CT scan with ¹⁸F-fluorodeoxyglucose (¹⁸F-FDG) after the administration of CAR-T cells and prednisone.

FIG. 3 shows transgene copies of CAR-T cells based on days post infusion.

FIG. 4 shows the number of cells per mL based on days post infusion.

FIG. 5A shows modulation of CAR-T expansion during prednisone treatment. The transgene copies of CAR-T cells and the CAR copy number are shown based on days post infusion. FIG. 5B shows CAR transgene copy numbers up to 90 days post infusion. Transgene copy numbers are detected using two different analytical methods: Digital droplet PCR (ddPCR) and quantitative PCR.

FIG. 6A depicts the quantitative flow cytometric analysis of specific cell populations detected in the blood at the sampled time points. FIG. 6B depicts the quantitative flow cytometric analysis of specific cell populations detected in the blood at the sampled time points.

FIG. 7A shows serum cytokine levels at different time points after CAR-T cells infusion. FIG. 7B shows serum cytokine levels at different time points after CAR-T cells infusion.

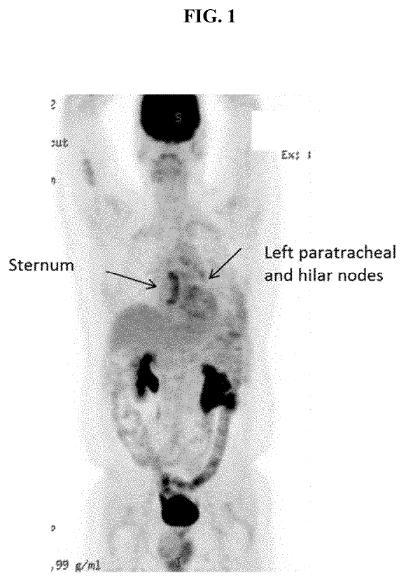

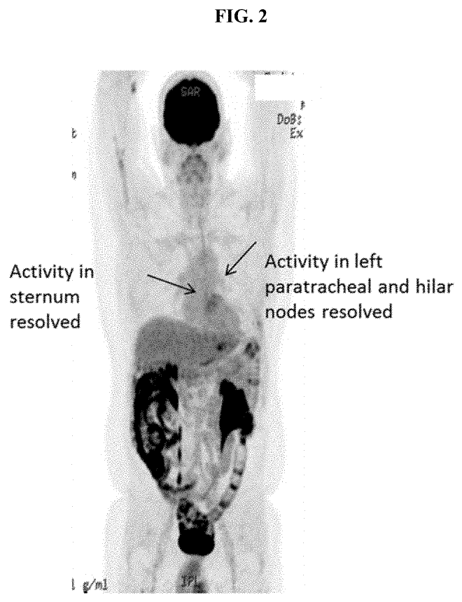

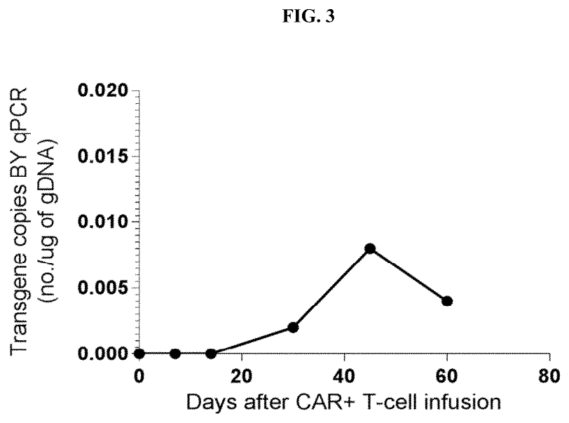

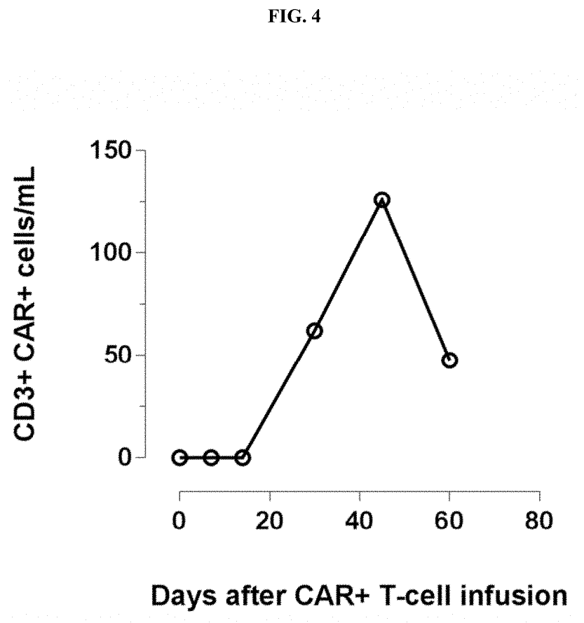

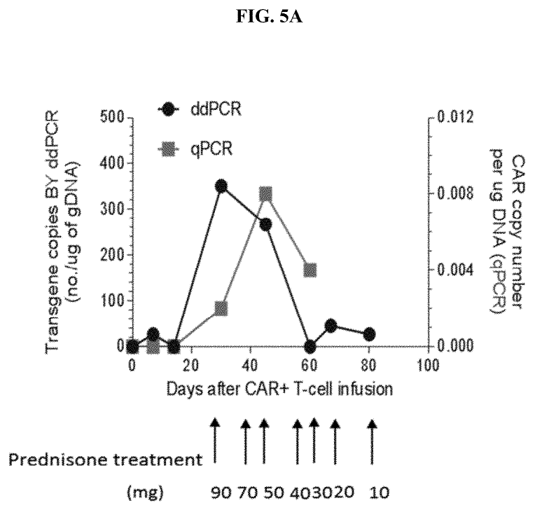

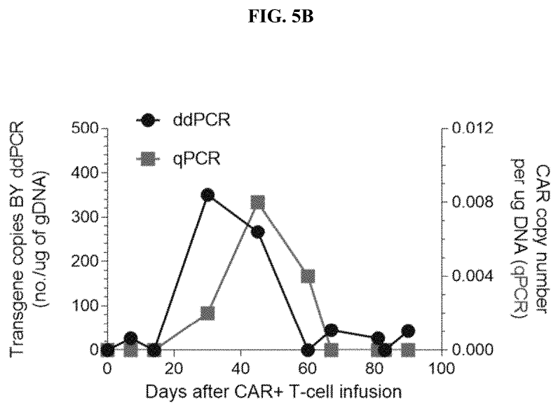

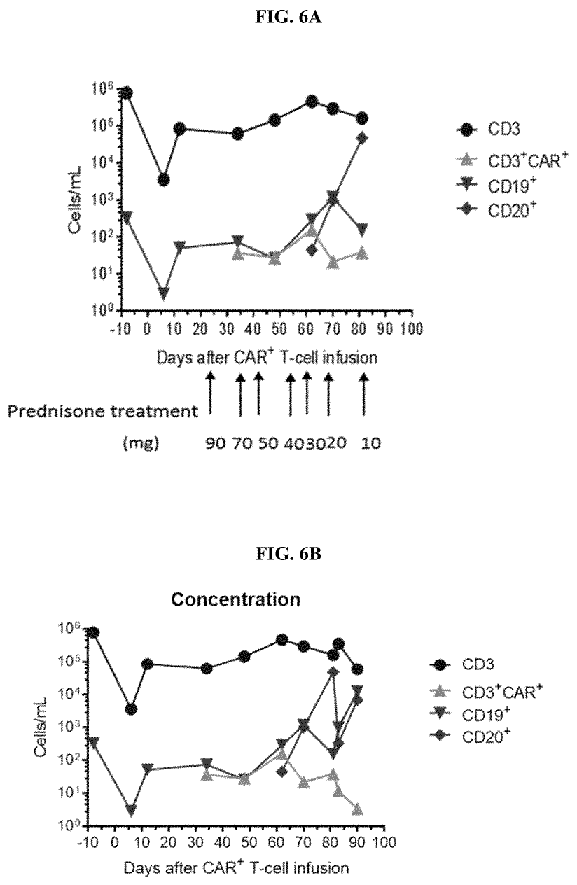

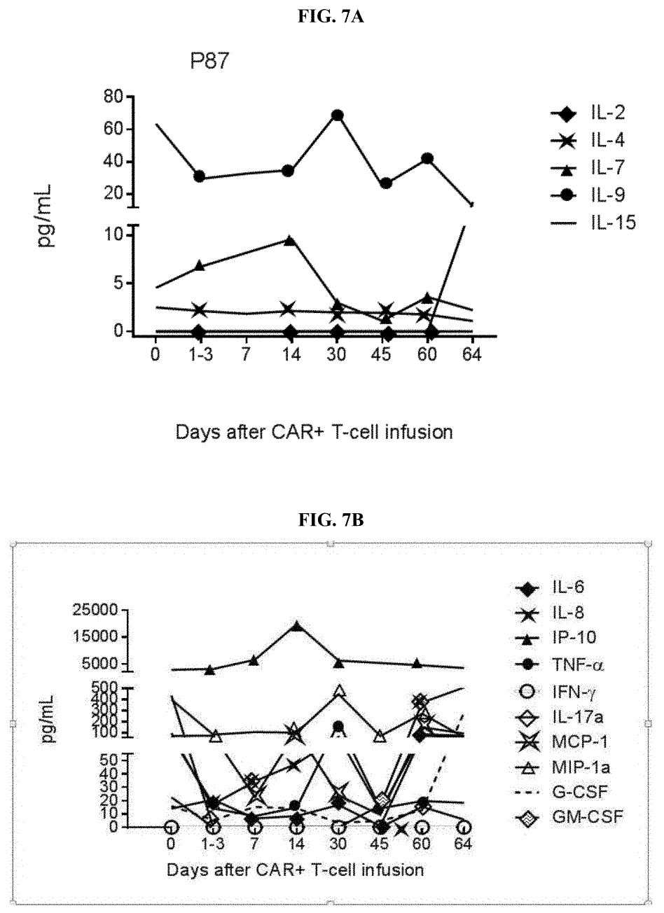

### DETAILED DESCRIPTION OF THE DISCLOSURE

Provided are methods and compositions including modified effector cells to target specific antigens and providing at least one steroid in an amount sufficient not to interfere with engraftment and/or sustained expansion of population of said modified effector cells. In some cases, the steroid is provided prior to the manifestation of any symptoms of a cytokine storm. In some cases, the steroid is provided prior to the administration of the modified effector cells to a subject in need thereof.

Chimeric antigen receptor (CAR) therapy comprises modified effector cells (e.g., T cells) to target specific tumor antigens. In some instances, CAR therapy has been used to target CD19, a pan-B cell antigen expressed in most B-cell malignancies. CD19-specific CAR therapy has shown efficacy in several clinical trials, for example, targeting acute lymphoblastic leukemia (ALL), chronic lymphocytic leukemia (CLL), or B-cell non-Hodgkin lymphoma (NHL). However, limited in vivo expansion of modified effector cells and low levels of persistence of these modified cells after administration have also been observed.

Disclosed herein, in certain embodiments, are methods of inducing expansion of a population of modified effector cells in a subject in need thereof. In some instances, disclosed herein include a method of administering to a subject a modified effector cell and a steroid, in which the steroid is in an amount effective not to adversely impact engraftment and/or expansion of the population of the modified effector cell in the subject.

In some instances, also disclosed herein is a method of inducing T cell expansion in a subject, which comprises contacting a T cell ex vivo with a vector encoding a chimeric antigen receptor to generate a CAR-T cell, administering to the subject an amount of the CAR-T cell, and administering to the subject a steroid in an amount sufficient to induce expansion of the population of CAR-T cells in the subject.

In some instances, additionally disclosed herein is a method of inducing T cell expansion in vivo, which comprises contacting a population of modified T cells in vivo with a first amount of steroid at a first time point not to adversely impact engraftment and/or expansion of the modified T cells, wherein each modified T cell comprises a chimeric receptor expressed on the cell surface.

In some instances, methods described herein enable the omission of a pre-conditioning step (e.g., lymphodepletion). In such cases, modified effector cells and a steroid described herein are administered to a subject without the need of a prior administration of a chemotherapeutic agent to induce lymphopenia. In some instances, the pre-conditioning step is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 days prior to administration of modified effector cells to the subject.

In additional instances, methods described herein comprise administration of a low dose of a steroid. In some instances, the low dose comprises a dose that is less than the dose needed for a reduction in at least one symptom of cytokine-associated toxicity. In some cases, the cytokine-associated toxicity comprises cytokine release syndrome (CRS), encephalopathy or B-cell aplasia. In further instances, the steroid is first administered at a certain dose that is subsequently successively reduced, e.g. the first dose is 1 mg/kg, the subsequent doses are 0.9 mg/kg, 0.8 mg/kg, 0.7 mg/kg, 0.6 mg/kg, 0.5 mg/kg, 0.4 mg/kg, 0.3 mg/kg, 0.2 mg/kg or 0.1 mg/kg.

### Modified Effector Cells

In some embodiments, modified effector cells are modified immune cells that comprise T cells and/or natural killer cells. T cells or T lymphocytes are a subtype of white blood cells that are involved in cell-mediated immunity. Exemplary T cells include T helper cells, cytotoxic T cells, TH17 cells, stem memory T cells (T_{SCM}), naïve T cells, memory T cells, effector T cells, regulatory T cells, or natural killer T cells.

T helper cells (TH cells) assist other white blood cells in immunologic processes, including maturation of B cells into plasma cells and memory B cells, and activation of cytotoxic T cells and macrophages. In some instances, TH cells are known as CD4+ T cells due to expression of the CD4 glycoprotein on the cell surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, Th9, or TFH, which secrete different cytokines to facilitate different types of immune responses. Signaling from the APC directs T cells into particular subtypes.

Cytotoxic T cells (TC cells or CTLs) destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise subtypes: stem memory T cells (T_{SCM}), central memory T cells (T_{CM} cells) and two types of effector memory T cells (T_{EM} cells and T_{EMRA} cells). Memory cells may be either CD4+ or CD8+. Memory T cells may express the cell surface proteins CD45RO, CD45RA and/or CCR7.

Regulatory T cells (Treg cells), formerly known as suppressor T cells, play a role in the maintenance of immunological tolerance. Their major role is to shut down T cell-mediated immunity toward the end of an immune reaction and to suppress autoreactive T cells that escaped the process of negative selection in the thymus.

Natural killer T cells (NKT cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

Natural killer (NK) cells are a type of cytotoxic lymphocyte of the innate immune system. In some instances, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

### Chimeric Receptors

In some embodiments, described herein includes a modified effector cell which comprises a chimeric receptor expressed on the surface of the cell. In some instances, the chimeric receptor comprises an antigen binding region that enables recognition and binding to a tumor antigen, e.g., a tumor-associated antigen or a tumor-specific antigen. In some instances, the antigen binding region comprises an antibody or binding fragment, for example, an Fab, an Fab′, an F(ab′)₂, an F(ab′)₃, an scFv, an sc(Fv)₂, a dsFv, a diabody, a minibody, and a nanobody or binding fragments thereof. In some cases, the antigen binding region comprises an scFv. In some cases, the chimeric receptor comprises an scFv (e.g., a chimeric antigen receptor (CAR)). In some instances, the chimeric antigen receptor comprises a pattern-recognition receptor. In other cases, the chimeric receptor comprises an engineered T-cell receptor (TCR).

### Chimeric Antigen Receptors (CARs)

A chimeric antigen receptor (CAR) is an engineered receptor which grafts an exogenous specificity onto an immune effector cell. In some instances, a CAR comprises an extracellular domain (ectodomain) that comprises an antigen binding domain, a stalk region, a transmembrane domain and an intracellular (endodomain) domain. In some instances, the intracellular domain further comprises one or more intracellular signaling domains. In some instances, a CAR described herein comprises an antigen binding domain, a stalk region, a transmembrane domain, one or more costimulatory domains, and a signaling domain for T-cell activation.

An antigen binding domain can comprise complementary determining regions of a monoclonal antibody, variable regions of a monoclonal antibody, and/or antigen binding fragments thereof. A complementarity determining region (CDR) is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor can contain three CDRs (CDR1, CDR2, and CDR3). In some instances, an antigen binding domain comprises F(ab′)₂, Fab′, Fab, Fv, or scFv. In some cases, an antigen binding domain is a scFv. In some cases, an antigen binding domain is a Fab. In some cases, an antigen binding domain is a Fab′. In some cases, an antigen binding domain is F(ab′)₂. In some cases, an antigen binding domain is a Fv.

In some embodiments, a CAR described herein comprises an antigen binding domain that binds to an epitope on CD19, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, Folate receptor a, Mucins such as MUC-1 or MUC-16, MAGE-A1, h5T4, PSMA, TAG-72, EGFR, CD20, EGFRvIII, CD123 or VEGF-R2. In some embodiments, a CAR described herein comprises an antigen binding domain that binds to an epitope on CD19 or CD33. In some instances, a CAR described herein comprises an antigen binding domain that binds to an epitope on CD19. In some cases, a CAR described herein comprises an antigen binding domain that binds to an epitope on CD33. In further embodiments, a CAR described herein comprises an autoantigen or an antigen binding region that binds to an epitope on HLA-A2, myelin oligodendrocyte glycoprotein (MOG), factor VIII (FVIII), MAdCAM1, SDF1, or collagen type II

In some embodiments, the CARs and methods described herein can be used for the treatment of a hyperproliferative disease, such as a cancer, an autoimmune disease or for the treatment of an infection, such as a viral, bacterial or parasitic infection. In some aspects, the CAR targets an antigen that is elevated in cancer cells, in autoimmune cells or in cells that are infected by a virus, bacteria or parasite. Pathogens that may be targeted include, without limitation, Plasmodium, trypanosome, Aspergillus, Candida, Hepatitis A, Hepatitis B, Hepatitis C, HSV, HPV, RSV, EBV, CMV, JC virus, BK virus, or Ebola pathogens. Autoimmune diseases can include graft-versus-host disease, rheumatoid arthritis, lupus, celiac disease, Crohn's disease, Sjogren Syndrome, polymyalgia rheumatic, multiple sclerosis, neuromyelitis optica, ankylosing spondylitis, Type 1 diabetes, alopecia areata, vasculitis, temporal arteritis, bullous pemphigoid, psoriasis, pemphigus vulgaris, or autoimmune uveitis.

The pathogen recognized by a CAR may be essentially any kind of pathogen, but in some embodiments the pathogen is a fungus, bacteria, or virus. Exemplary viral pathogens include those of the families of Adenoviridae, Epstein-Barr virus (EBV), Cytomegalovirus (CMV), Respiratory Syncytial Virus (RSV), JC virus, BK virus, HPV, HSV, HHV family of viruses, Hepatitis family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In some embodiments the pathogen receptor Dectin-1 may be used to generate a CAR that recognizes the carbohydrate structure on the cell wall of fungi such as Aspergillus. In another embodiment, CARs can be made based on an antibody recognizing viral determinants (e.g., the glycoproteins from CMV and Ebola) to interrupt viral infections and pathology.

In some embodiments, a “stalk” region, or a “spacer” or “hinge” region, is used to link the antigen-binding domain to the transmembrane domain. In some instances, a “stalk domain” or “stalk region” comprise any oligonucleotide- or polypeptide that functions to link the transmembrane domain to, either the extracellular domain or, the cytoplasmic domain in the polypeptide chain. In some embodiments, it is flexible enough to allow the antigen-binding domain to orient in different directions to facilitate antigen recognition. In some instances, the stalk region comprises the hinge region from IgG1. In alternative instances, the stalk region comprises the CH2CH3 region of immunoglobulin and optionally portions of CD3. In some cases, the stalk region comprises a CD8a hinge region, an IgG4-Fc 12 amino acid hinge region (ESKYGPPCPPCP) or IgG4 hinge regions as described in WO/2016/073755.

The transmembrane domain can be derived from either a natural or a synthetic source. Where the source is natural, the domain can be derived from any membrane-bound or transmembrane protein. Suitable transmembrane domains can include the transmembrane region(s) of alpha, beta or zeta chain of the T-cell receptor, or a transmembrane region from CD28, CD3 epsilon, CD3ζ, CD45, CD4, CD5, CD8alpha, CD9, CD16, CD22, CD33, CD37, CD64, CD80, CD86, CD134, CD137 or CD154. Alternatively the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the cytoplasmic signaling domain of a CAR. In some embodiments, the linker is a glycine-serine linker.

In some embodiments, the transmembrane domain comprises a CD8a transmembrane domain or a CD3ζ transmembrane domain. In some embodiments, the transmembrane domain comprises a CD8α transmembrane domain. In other embodiments, the transmembrane domain comprises a CD3ζ transmembrane domain.

The intracellular domain can comprise one or more costimulatory domains. Exemplary costimulatory domains include, but are not limited to, CD8, CD27, CD28, 4-1BB (CD137), ICOS, DAP10, DAP12, OX40 (CD134) or fragment or combination thereof. In some instances, a CAR described herein comprises one or more, or two or more of costimulatory domains selected from CD8, CD27, CD28, 4-1BB (CD137), ICOS, DAP10, DAP12, OX40 (CD134) or fragment or combination thereof. In some instances, a CAR described herein comprises one or more, or two or more of costimulatory domains selected from CD27, CD28, 4-1BB (CD137), ICOS, OX40 (CD134) or fragment or combination thereof. In some instances, a CAR described herein comprises one or more, or two or more of costimulatory domains selected from CD8, CD28, 4-1BB (CD137), or fragment or combination thereof. In some instances, a CAR described herein comprises one or more, or two or more of costimulatory domains selected from CD28, 4-1BB (CD137), or fragment or combination thereof. In some instances, a CAR described herein comprises costimulatory domains CD28 and 4-1BB (CD137) or their respective fragments thereof. In some instances, a CAR described herein comprises costimulatory domains CD28 and OX40 (CD134) or their respective fragments thereof. In some instances, a CAR described herein comprises costimulatory domains CD8 and CD28 or their respective fragments thereof. In some instances, a CAR described herein comprises costimulatory domains CD28 or a fragment thereof. In some instances, a CAR described herein comprises costimulatory domains 4-1BB (CD137) or a fragment thereof. In some instances, a CAR described herein comprises costimulatory domains OX40 (CD134) or a fragment thereof. In some instances, a CAR described herein comprises costimulatory domains CD8 or a fragment thereof.

In some embodiments, the intracellular domain further comprises a signaling domain for T-cell activation. In some instances, the signaling domain for T-cell activation comprises a domain derived from TCR zeta, FcR gamma, FcR beta, CD3 gamma, CD3 delta, CD3 epsilon, CD5, CD22, CD79a, CD79b or CD66d. In some cases, the signaling domain for T-cell activation comprises a domain derived from CD3ζ.

### CD19-Specific CARs

CD19 is a cell surface glycoprotein of the immunoglobulin superfamily and is found predominately in malignant B-lineage cells. In some instances, CD19 has also been detected in solid tumors such as pancreatic cancer, liver cancer, and prostate cancer.

In some embodiments, described herein include a CD19-specific CAR, in which the antigen binding domain comprises a F(ab′)₂, Fab′, Fab, Fv, or scFv. In some instances, the antigen binding domain recognizes an epitope on CD19.

In some embodiments, the antigen binding domain recognizes an epitope on CD9 that is also recognized by JCAR014. JCAR015. JCAR017, or 19-28z CAR (Juno Therapeutics). In some embodiments, described herein include a CD19-specific CAR-T cell, in which the antigen binding domain recognizes an epitope on CD19 that is also recognized by JCAR014, JCAR015, JCAR017, or 19-28z CAR (Juno Therapeutics). In some instances, the CD19-specific CAR-T cell further comprises a transmembrane domain selected from a CD8alpha transmembrane domain or a CD3ζ transmembrane domain; one or more costimulatory domains selected from CD27, CD28, 4-1BB (CD137), ICOS, DAP10, OX40 (CD134) or fragment or combination thereof; and a signaling domain from CD3ζ.

In some embodiments, described herein include a CD19-specific CAR-T cell comprises a scFv antigen binding domain, and the antigen binding domain recognizes an epitope on CD19 that is also recognized by JCAR014, JCAR015, JCAR017, or 19-28z CAR (Juno Therapeutics). In some instances, the CD19-specific CAR-T cell further comprises a transmembrane domain selected from a CD8alpha transmembrane domain or a CD3ζ transmembrane domain; one or more costimulatory domains selected from CD27, CD28, 4-1BB (CD137), ICOS, DAP10, OX40 (CD134) or fragment or combination thereof; and a signaling domain from CD3ζ.

In some embodiments, a CD19-specific CAR-T cell described herein comprises an anti-CD19 antibody described in US20160152723.

In some embodiments, the antigen binding domain recognizes an epitope on CD19 that is also recognized by KTE-C19 (Kite Pharma. Inc.). In some embodiments, described herein include a CD19-specific CAR-T cell, in which the antigen binding domain recognizes an epitope on CD19 that is also recognized by KTE-C19. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, OX40 (CD134) or fragment or combination thereof; and a signaling domain from CD3ζ.

In some embodiments, described herein include a CD19-specific CAR-T cell comprises a scFv antigen binding domain, and the antigen binding domain recognizes an epitope on CD19 that is also recognized by KTE-C19. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, a CD19-specific CAR-T cell described herein comprises an anti-CD19 antibody described in WO2015187528 or fragment or derivative thereof.

In some embodiments, the antigen binding domain recognizes an epitope on CD19 that is also recognized by CTL019 (Novartis). In some embodiments, described herein include a CD19-specific CAR-T cell, in which the antigen binding domain recognizes an epitope on CD19 that is also recognized by CTL019. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, described herein include a CD19-specific CAR-T cell comprises a scFv antigen binding domain, and the antigen binding domain recognizes an epitope on CD19 that is also recognized by CTL019. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, the antigen binding domain recognizes an epitope on CD19 that is also recognized by UCART19 (Cellectis). In some embodiments, described herein include a CD19-specific CAR-T cell, in which the antigen binding domain recognizes an epitope on CD19 that is also recognized by UCART19. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, described herein include a CD19-specific CAR-T cell comprises a scFv antigen binding domain, and the antigen binding domain recognizes an epitope on CD19 that is also recognized by UCART19. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, the antigen binding domain recognizes an epitope on CD19 that is also recognized by BPX-401 (Bellicum). In some embodiments, described herein include a CD19-specific CAR-T cell, in which the antigen binding domain recognizes an epitope on CD19 that is also recognized by BPX-401. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some embodiments, described herein include a CD19-specific CAR-T cell comprises a scFv antigen binding domain, and the antigen binding domain recognizes an epitope on CD19 that is also recognized by BPX-401. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some cases, the antigen binding domain recognizes an epitope on CD19 that is also recognized by blinatumomab (Amgen), coltuximabravtansine (ImmunoGen Inc./Sanofi-aventis), MOR208 (Morphosys AG/Xencor Inc.). MEDI-551 (Medimmune), denintuzumabmafodotin (Seattle Genetics), B4 (or DI-B4) (Merck Serono), taplitumomabpaptox (National Cancer Institute), XmAb 5871 (AmgeniXencor, Inc.), MDX-1342 (Medarex) or AFM11 (Affimed). In some instances, the CD19-specific CAR further comprises a transmembrane domain selected from a CD8alpha transmembrane domain or a CD3ζ transmembrane domain; one or more costimulatory domains selected from CD27, CD28, 4-1BB (CD137), ICOS, DAP10, OX40 (CD134) or fragment or combination thereof; and a signaling domain from CD3ζ.

In some embodiments, described herein include a CD19-specific CAR-T cell, in which the antigen binding domain comprises a F(ab′)₂, Fab′, Fab, Fv, or scFv. In some instances, the antigen binding domain recognizes an epitope on CD19. In some cases, the antigen binding domain recognizes an epitope on CD19 that is also recognized by blinatumomab (Amgen), coltuximabravtansine (ImmunoGen Inc./Sanofi-aventis), MOR208 (Morphosys AG/Xencor Inc.), MEDI-551 (Medimmune), denintuzumabmafodotin (Seattle Genetics), B4 (or DI-B4) (Merck Serono), taplitumomabpaptox (National Cancer Institute), XmAb 5871 (Amgen/Xencor, Inc.). MDX-1342 (Medarex) or AFM11 (Affimed). In some instances, the CD19-specific CAR-T cell further comprises a transmembrane domain selected from a CD8alpha transmembrane domain or a CD3ζ transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

In some cases, a CD19-specific CAR-T cell described herein comprise a scFv antigen binding domain, and the antigen binding domain recognizes an epitope on CD19 that is also recognized by blinatumomab (Amgen), coltuximabravtansine (ImmunoGen Inc./Sanofi-aventis), MOR208 (Morphosys AG/Xencor Inc.), MEDI-551 (Medimmune), denintuzumabmafodotin (Seattle Genetics), B4 (or DI-B4) (Merck Serono), taplitumomabpaptox (National Cancer Institute), XmAb 5871 (AmgeniXencor, Inc.), MDX-1342 (Medarex) or AFM11 (Affimed). In some instances, the CD19-specific CAR-T cell further comprises a transmembrane domain selected from a CD8alpha transmembrane domain or a CD3ζ transmembrane domain; one or more costimulatory domains selected from CD27, CD28, 4-1BB (CD137), ICOS, DAP10, DAP12, OX40 (CD134) or fragment or combination thereof; and a signaling domain from CD3ζ.

### Engineered T-cell Receptor (TCR)

In some embodiments, the chimeric receptor comprises an engineered T-cell receptor. The T cell receptor (TCR) is composed of two chains (αβ or γδ) that pair on the surface of the T cell to form a heterodimeric receptor. In some instances, the αβ TCR is expressed on most T cells in the body and is known to be involved in the recognition of specific MHC-restricted antigens. Each α and β chain are composed of two domains: a constant domain (C) which anchors the protein to the cell membrane and is associated with invariant subunits of the CD3 signaling apparatus; and a variable domain (V) that confers antigen recognition through six loops, referred to as complementarity determining regions (CDRs). In some instances, each of the V domains comprises three CDRs; e.g., CDR1, CDR2 and CDR3 with CDR3 as the hypervariable region. These CDRs interact with a complex formed between an antigenic peptide bound to a protein encoded by the major histocompatibility complex (pepMHC) (e.g., HLA-A, HLA-B, HLA-C. HLA-DPA1, HLA-DPB1, HLA-DQA1, HLA-DQB1, HLA-DRA, or HLA-DRB1 complex). In some instances, the constant domain further comprises a joining region that connects the constant domain to the variable domain. In some cases, the beta chain further comprises a short diversity region which makes up part of the joining region.

In some cases, such TCR are reactive to specific tumor antigen, e.g. NY-ESO, Mage A3, Titin. In other cases, such TCR are reactive to specific neoantigens expressed within a patient's tumor (i.e. patient-specific, somatic, non-synonymous mutations expressed by tumors). In some cases, engineered TCRs can be affinity-enhanced.

In some embodiments, a TCR is described using the International Immunogenetics (IMGT) TCR nomenclature, and links to the IMGT public database of TCR sequences. For example, there can be several types of alpha chain variable (Va) regions and several types of beta chain variable (Vβ) regions distinguished by their framework, CDR1, CDR2, and CDR3 sequences. As such, a Vα type can be referred to in IMGT nomenclature by a unique TRAV number. For example, “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV5-1” defines a TCR Vβ region having unique framework and CDR1 and CDR2 sequences, but with only a partly defined CDR3 sequence.

In some cases, the beta chain diversity region is referred to in IMGT nomenclature by the abbreviation TRBD.

In some instances, the unique sequences defined by the IMGT nomenclature are widely known and accessible to those working in the TCR field. For example, they can be found in the IMGT public database and in “T cell Receptor Factsbook”, (2001) LeFranc and LeFranc, Academic Press, ISBN 0-12-441352-8.

In some embodiments, an αβ heterodimeric TCR is, for example, transfected as full length chains having both cytoplasmic and transmembrane domains. In some cases, the TCRs contain an introduced disulfide bond between residues of the respective constant domains, as described, for example, in WO 2006/000830.

In some instances, TCRs described herein are in single chain format, for example see WO 2004/033685. Single chain formats include αβ TCR polypeptides of the Vα-L-V, Vβ-L-Vα, Vα-Cα-L-V, Vα-L-Vβ-Cβ, Vα-Cα-L-Vβ-Cβ types, wherein Vα and Vβ are TCR α and β variable regions respectively, Cα and Cβ are TCR α and β constant regions respectively, and L is a linker sequence. In certain embodiments single chain TCRs of the invention may have an introduced disulfide bond between residues of the respective constant domains, as described in WO 2004/033685.

The TCR described herein may be associated with a detectable label, a therapeutic agent or a PK modifying moiety.

Exemplary detectable labels for diagnostic purposes include, but are not limited to, fluorescent labels, radiolabels, enzymes, nucleic acid probes and contrast reagents.

Therapeutic agents which may be associated with the TCRs described herein include immunomodulators, radioactive compounds, enzymes (perforin for example) or chemotherapeutic agents. To ensure that toxic effects are exercised in the desired location the toxin could be inside a liposome linked to a TCR so that the compound is released in a controlled manner. In some cases, the controlled release minimize damaging effects during the transport in the body and ensure that the toxin has maximum effect after binding of the TCR to the relevant antigen presenting cells.

In some embodiments, additional suitable therapeutic agents include for instance:
a. small molecule cytotoxic agents, e.g., compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin;
b. peptide cytotoxins, i.e. proteins or fragments thereof with the ability to kill mammalian cells. For example, ricin, diphtheria toxin, Pseudomonas bacterial exotoxin A, DNase and RNase;
c. radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
d. immuno-stimulants, i.e. immune effector molecules which stimulate immune response. For example, cytokines such as IL-2 and IFN-γ,
e. superantigens and mutants thereof;
f. TCR-HLA fusions;
g. chemokines such as IL-8, platelet factor 4, melanoma growth stimulatory protein, etc;
h. antibodies or fragments thereof, including anti-T cell or NK cell determinant antibodies (e.g. anti-CD3, anti-CD28 or anti-CD16);
i. alternative protein scaffolds with antibody like binding characteristics
j. complement activators; and
k. xenogeneic protein domains, allogeneic protein domains, viral/bacterial protein domains, viral/bacterial peptides.

### Modified Effector Cell Doses

In some embodiments, an amount of modified effector cells is administered to a subject in need thereof and the amount is determined based on the efficacy and the potential of inducing a cytokine-associated toxicity. In some cases, an amount of modified effector cells comprises about 10⁵ to about 10⁹ modified effector cells/kg. In some cases, an amount of modified effector cells comprises about 10⁵ to about 10⁵ modified effector cells/kg. In some cases, an amount of modified effector cells comprises about 10⁵ to about 10⁷ modified effector cells/kg. In some cases, an amount of modified effector cells comprises about 10⁶ to about 10⁹ modified effector cells/kg. In some cases, an amount of modified effector cells comprises about 10⁶ to about 10l modified effector cells/kg. In some cases, an amount of modified effector cells comprises about 10⁷ to about 10⁹ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some instances, an amount of modified effector cells comprises about 10⁹ modified effector cells/kg. In some instances, an amount of modified effector cells comprises about 10⁸ modified effector cells/kg. In some instances, an amount of modified effector cells comprises about 10⁷ modified effector cells/kg. In some instances, an amount of modified effector cells comprises about 10⁶ modified effector cells/kg. In some instances, an amount of modified effector cells comprises about 10⁵ modified effector cells/kg.

In some embodiments, the modified effector cells are modified T cells. In some instances, the modified T cells are CAR-T cells. In some cases, an amount of CAR-T cells comprises about 10⁵ to about 10⁹ CAR-T cells/kg. In some cases, an amount of CAR-T cells comprises about 10⁵ to about 10⁸ CAR-T cells/kg. In some cases, an amount of CAR-T cells comprises about 10⁵ to about 10⁷ CAR-T cells/kg. In some cases, an amount of CAR-T cells comprises about 10⁶ to about 10⁹ CAR-T cells/kg. In some cases, an amount of CAR-T cells comprises about 10⁶ to about 10⁸ CAR-T cells/kg. In some cases, an amount of CAR-T cells comprises about 10⁷ to about 10⁹ CAR-T cells/kg. In some cases, an amount of CAR-T cells comprises about 10⁵ to about 10⁶ CAR-T cells/kg. In some cases, an amount of CAR-T cells comprises about 10⁶ to about 10⁷ CAR-T cells/kg. In some cases, an amount of CAR-T cells comprises about 10⁷ to about 10⁸ CAR-T cells/kg. In some cases, an amount of CAR-T cells comprises about 10⁸ to about 10⁹ CAR-T cells/kg. In some instances, an amount of CAR-T cells comprises about 10⁹ CAR-T cells/kg. In some instances, an amount of CAR-T cells comprises about 10⁸ CAR-T cells/kg. In some instances, an amount of CAR-T cells comprises about 10⁷ CAR-T cells/kg. In some instances, an amount of CAR-T cells comprises about 10⁶ CAR-T cells/kg. In some instances, an amount of CAR-T cells comprises about 10⁵ CAR-T cells/kg.

In some embodiments, the CAR-T cells are CD19-specific CAR-T cells. In some cases, an amount of CD19-specific CAR-T cells comprises about 10⁵ to about 10⁹ CAR-T cells/kg. In some cases, an amount of CD19-specific CAR-T cells comprises about 10⁵ to about 10⁸ CAR-T cells/kg. In some cases, an amount of CD19-specific CAR-T cells comprises about 10⁵ to about 10⁷ CAR-T cells/kg. In some cases, an amount of CD19-specific CAR-T cells comprises about 10⁶ to about 10⁹ CAR-T cells/kg. In some cases, an amount of CD19-specific CAR-T cells comprises about 10⁶ to about 10⁸ CAR-T cells/kg. In some cases, an amount of CD19-specific CAR-T cells comprises about 10⁷ to about 10⁹ CAR-T cells/kg. In some cases, an amount of CD19-specific CAR-T cells comprises about 10⁵ to about 10⁶ CAR-T cells/kg. In some cases, an amount of CD19-specific CAR-T cells comprises about 10⁶ to about 10⁷ CAR-T cells/kg. In some cases, an amount of CD19-specific CAR-T cells comprises about 10⁷ to about 10⁸ CAR-T cells/kg. In some cases, an amount of CD19-specific CAR-T cells comprises about 10⁸ to about 10⁹ CAR-T cells/kg. In some instances, an amount of CD19-specific CAR-T cells comprises about 10⁹ CAR-T cells/kg. In some instances, an amount of CD19-specific CAR-T cells comprises about 10⁸ CAR-T cells/kg. In some instances, an amount of CD19-specific CAR-T cells comprises about 10⁷ CAR-T cells/kg. In some instances, an amount of CD19-specific CAR-T cells comprises about 10⁶ CAR-T cells/kg. In some instances, an amount of CD19-specific CAR-T cells comprises about 10⁵ CAR-T cells/kg.

In some embodiments, the modified T cells are engineered TCR T-cells. In some cases, an amount of engineered TCR T-cells comprises about 10⁵ to about 10⁹ TCR cells/kg. In some cases, an amount of engineered TCR cells comprises about 10⁵ to about 10 TCR cells/kg. In some cases, an amount of engineered TCR cells comprises about 10⁵ to about 10⁷ TCR cells/kg. In some cases, an amount of engineered TCR cells comprises about 10⁶ to about 10⁹ TCR cells/kg. In some cases, an amount of engineered TCR cells comprises about 10⁶ to about 10⁸ TCR cells/kg. In some cases, an amount of engineered TCR cells comprises about 10⁷ to about 10⁹ TCR cells/kg. In some cases, an amount of engineered TCR cells comprises about 10⁵ to about 10⁶ TCR cells/kg. In some cases, an amount of engineered TCR cells comprises about 10⁶ to about 10⁷ TCR cells/kg. In some cases, an amount of engineered TCR cells comprises about 10⁷ to about 10⁸ TCR cells/kg. In some cases, an amount of engineered TCR cells comprises about 10⁸ to about 10⁹ TCR cells/kg. In some instances, an amount of engineered TCR cells comprises about 10⁹ TCR cells/kg. In some instances, an amount of engineered TCR cells comprises about 10⁸ TCR cells/kg. In some instances, an amount of engineered TCR cells comprises about 10⁷ TCR cells/kg. In some instances, an amount of engineered TCR cells comprises about 10⁶ TCR cells/kg. In some instances, an amount of engineered TCR cells comprises about 10⁵ TCR cells/kg.

### Steroids

Steroids, such as androgens, corticosteroids, estrogens and progestogens control many physiological processes in humans including reproduction, secondary sexual characteristics, maturation, gene expression, cardiovascular health and neurological functions. In some instances, a steroid is further divided based on either a structural classification or a functional classification. Structural classification can divide steroids into six classes: gonane (having 17 carbons), estrane (having 18 carbons), androstane (having 19 carbons), pregnane (having 21 carbons), cholane (having 24 carbons), and cholestane (having 27 carbons). Functional classification can divide steroids into five classes: estrogens (having 18 carbons), androgens (having 19 carbons), corticosteroids (having 21 carbons), progesterone (having 21 carbons) and sterols (having 27 carbons).

Exemplary steroids include, but are not limited to, 17-hydroxypregnenolone; androstenolone (DHEA); 5-androstenediol; progesterone; 17-h.ydroxyprogest.erone; eortexolone; Cortisol; eortexone; corticosterone; aldosterone; androstenedione; testosterone; allodihydrotestosterone; estrone E1; estradiol E2; 11a-hydroxyestrone; 11b-hydroxyestrone; I 1a-hydroxyestradiol; 11b-hydroxyestradiol; 9(11)-dehydroestradiol; 9(11)-dehydroestrone; 11-ketoestrone; estradiol-3-SO₄; estrone-SO₄; estradiol-17-SO₄; estradiol-3, 17-di-SO₄; 3-OM estrone; estradiol-3-Glu; estrone-Giu; estradiol-17-Glu; estradiol-3, 17-di-Glu; 4-OH estrone; 4-OH estradiol; 4-OMe estone; 4-OMe estradiol; 4-OMe estriol; 2-OH estrone; 2-hydroxyestradiol; 2-GH eslril; 3-methoxy-2-OH-estrone; 2-methoxy-2-QH-estrone; 2-methoxy-3-OH-estradiol; 2,3-dimethoxyestrone; 2,3-dimethox estradiol; 6a-hydroxyestradiol; 6b-hydroxyestradiol; 6-ketostrone; 6-ketoestradiol; 6-dehydroestradiol; 6-dehydroestrone; 16a-hydroxyestrone; 17-epiestriol; estriol E3; 16,17-epiestriol; 16-epiestriol; estriol-3-sulfate; estrioL-17-Ghi; 3-methoxystriol; 16-keto-1.7b-estrad.iol; 6-ketoestrioi; 7-dehydro-17b-estradiol; equilin; dihydroequi!in-3-SO₄; equilin-3-SO₄; 3a-hydroxy-5a-pregnan-20-one; 5a-diiiydroprogesterone; Allopregnanediol; 11a-hydroxy-4-pregnene-3,20-dione; 11b-hydroxy-4-pregnene-3,20-dione; 1.7-hydroxypregnenolone; 17-hydroxyprogesterone; 2.1-hydroxypregnanolone; 20-hydroxypregneiiolone; 7a-hydroxypregnenoione; 2-GH-testosterone; 20a.-h.ydroxy-5a.-preg.na.n-3-one; 20a-dihydroprogesterone; 17a,20a-dihydroxyprogesterone; 3b-hydroxy-5-pregnen-20-one-3-SO₄; eltanolone; pregnanediol; 5b-dihydro progesterone; 5a-dihydrotestosterone; 17b-di.hydroandrost.erone; 17b-dihydroepiandrosterone; 7a-hydroxytestosterone; 7a-hydroxyandrostenedione; 6a-OH-testosterone; 6b-OH-testosterone; etiocholanolone glucuronide; DHEA GLUC; 16a-hydroxy DHEA; testosterone sulfate; desoxytestosterone; 4-androstene-30L, 17-one; 2a-liydroxyandrostenedione; 11a-hydroxyandrostenedione; 6-ketotesteroiie; NI 1-ketotestosterone; 9-dehydrotestosterone; allotetrahydrocortexone; adrenosterone; etiocholanolone; 4-androstenediol; 16a-ketotestosterone; 19-hydrox androstendione; 4-androsteti-3,6-17-trione; 7b-hydroxy DHEA; 7a-hydroxy DHEA; 7-ketoDHEA; 9-deiiydroepiaiidrosterone; aliochoiesterol; 9-dehydroprogesterone; 11-dehydrotetrahydrocorticosterone; tetrahydrocorticosterone; 6b-h.ydroxy DOC; urocortisol; pregnanetriol; 16a-hydroxyprogesterone; 17a-hydroxypregnenolone; 6-dehydrotestosterone; 9(11)-dehydro DHEA; 11b-hydroxyepiandrosterone; 16a-hydroxyepiandrosterone; androsterone; epiandrosterone; 7a-hydroxyandrostenediol (DHEA); 7b-hydroxyandrostenediol (DHEA); 4-cholestene-3a-OL; 4-pregnen-3b-ol-20-one; 5-androsten-3b,17-diol-16-one; 19-hydroxy DHEA; 16a-OH-pregnenolone; 5a-dihydrocortexone (5a-DH-DOC); epitestosterone; 5b-androstandione; 1b-OH-androstenedione; 1 l-keto-etiocholanolone; 5a-pregnan-11a-OH-3,20-dione; 5a-epoxypregnenolone; 5a-dihydro-11-keto-progesterone; 1-ketoprogesterone; cholesterol; 4b-OH cholesterol; 7a-OH cholesterol; 7b-OH cholesterol; 25-OH cholesterol; 5a,6a-epoxy cholesterol; 7-keto-cholesterol; 7-keto-25-OH cholesterol; 6,7-dehydrocholesterol; 6-dehydroch.olestenone; 6-ketoch.olestenone; etiocholan-3-OH-17-one; 7-dehydrocholesterol; dihydrocholesterol; cholesterol-3-S04; coprostano3-7,12-diol; 20a-OH-cholesterol; 22b-OH-cholesterol; 27-OH-cb.cholesterol; 24-keto-cholesterol; 24-hydroxycholesterol; Me-3OH-chol-5-n-24-oate; chenodeoxycholic acid; ursodeoxycholic acid; murocliolic acid; Iivodeoxycholic acid; allolitliocholic acid; lithocholic acid; taurocholanic acid; taurohyodeoxycholic acid; cholic acid; taurochenodeoxycholic acid; desoxvcholic acid; taurolitliocliolic acid; glycolithocholic acid; 6-keto-allolithocholic acid; glycodeoxycholic acid; glycochenodeoxycholic acid; tauroursodeoxycholic acid; taurodeoxycholic acid; glycodeoxycholic acid; a-muricholic acid; b-muricholic acid; tauro-b-muricholic acid; taurocholic acid; iaurodehydrocholic acid; giyeodehydrocholic acid; androstandiol-3-Glu; 11-keto-etiocholanolone-3-Glu; etiocholanolone-3-Glu; debydroepiandrosterone sulfate (DHEAS); epitestosterone-17-SO₄; testosterone-17-Glu; pregnanediol-3-Glu; 17-OH-pregnanolone-3-Glu-Na; pregnanolone-3-SO₄; 17,20-di-OH-progesterone-20-Glu; cortisol-21-SO₄; pregnenolone-3-Giu; cholesterol-3-Glu; allopregnanolone-SO₄; epiallopregnanolone-SO₄; estriol-3-Glu-Na; 24-dehydrocholesterol; coprosterol; campesterol; b-sitosterol; brassicateroi; stigmasterol; epialiochoiesterol; 24,25-epoxy cholesterol; zymosterol; lanosterol; lathosterol; 17β-estradiol; an analog thereof; or a derivative thereof.

In some embodiments, a steroid is an anabolic steroid (or an anabolic-androgenic steroid, AAS), a synthetic steroid that is structurally related to testosterone and further has similar effects as testosterone. Exemplary anabolic steroids include, but are not limited to, testosterone propionate, testosterone enanthate, testosterone cypionate, nandrolone (e.g., nandrolone decanoate or nandrolone phenylpropionate), stanozolol, methandienone (methandrostenolone), methyltestosterone, oxandrolone, mesterolone, oxymetholone, drostanolone, methenolone (methylandrostenolone), fluoxymesterone or a derivative thereof.

In some embodiments, a steroid is a corticosteroid. A corticosteroid can further be divided into glucocorticoids and mineralocorticoids. Exemplary corticosteroids include, but are not limited to, aldosterone, alclometasone dipropionate, amcinonide, betamethasone, betamethasone valerate, betamethasone dipropionate, budesonide, clobetasone-17-butyrate, clobetasol-17-propionate, cortisol (hydrocortisone), cortisone, cortisone acetate, dexamethasone, desonide, fludrocortisone, fluocinonide, fluocinolone acetonide, fluocortolone, fluocortolone caproate, fluocortolone pivalate, fluprednidene acetate, halcinonide, halometasone, hydrocortisone acetate, hydrocortisone-17-valerate, methylprednisolone, mometasone, prednicarbate, prednisolone, prednisone, tixocortol pivalate, triamcinolone acetonide, triamcinolone alcohol or a derivative thereof.

In some embodiments, a corticosteroid comprises a prodrug form. In some embodiments, a corticosteroid prodrug comprises hydrocortisone-17-butyrate, hydrocortisone-17-aceponate, hydrocortisone-17-buteprate, ciclesonide, or prednicarbate.

In some embodiments, disclosed herein include methods of administering to a subject a modified effector cell described supra and a steroid. In some cases, the steroid is administered in an amount effective to induce expansion of a population of a modified effector cell in vivo. In some instances, a steroid selected from testosterone propionate, testosterone enanthate, testosterone cypionate, nandrolone (e.g., nandrolone decanoate or nandrolone phenylpropionate), stanozolol, methandienone (methandrostenolone), methyltestosterone, oxandrolone, mesterolone, oxymetholone, drostanolone, methenolone (methylandrostenolone), fluoxymesterone, aldosterone, alclometasone dipropionate, amcinonide, betamethasone, betamethasone valerate, betamethasone dipropionate, budesonide, clobetasone-17-butyrate, clobetasol-17-propionate, cortisol (hydrocortisone), cortisone, cortisone acetate, dexamethasone, desonide, fludrocortisone, fluocinonide, fluocinolone acetonide, fluocortolone, fluocortolone caproate, fluocortolone pivalate, fluprednidene acetate, halcinonide, halometasone, hydrocortisone acetate, hydrocortisone-17-valerate, methylprednisolone, mometasone, prednicarbate, prednisolone, prednisone, tixocortol pivalate, triamcinolone acetonide, triamcinolone alcohol, hydrocortisone-17-butyrate, hydrocortisone-17-aceponate, hydrocortisone-17-buteprate, ciclesonide, or prednicarbate is administered with a modified effector cell described herein to a subject in need thereof, wherein the steroid is administered in an amount effective to induce expansion of a population of the modified effector cell in the subject. In some instances, a steroid selected from testosterone propionate, testosterone enanthate, testosterone cypionate, nandrolone (e.g., nandrolone decanoate or nandrolone phenylpropionate), stanozolol, methandienone (methandrostenolone), methyltestosterone, oxandrolone, mesterolone, oxymetholone, drostanolone, methenolone (methylandrostenolone), fluoxymesterone is administered with a modified effector cell described herein to a subject in need thereof, wherein the steroid is administered in an amount effective to induce expansion of a population of the modified effector cell in the subject. In some instances, a steroid selected from aldosterone, alclometasone dipropionate, amcinonide, betamethasone, betamethasone valerate, betamethasone dipropionate, budesonide, clobetasone-17-butyrate, clobetasol-17-propionate, cortisol (hydrocortisone), cortisone, cortisone acetate, dexamethasone, desonide, fludrocortisone, fluocinonide, fluocinolone acetonide, fluocortolone, fluocortolone caproate, fluocortolone pivalate, fluprednidene acetate, halcinonide, halometasone, hydrocortisone acetate, hydrocortisone-17-valerate, methylprednisolone, mometasone, prednicarbate, prednisolone, prednisone, tixocortol pivalate, triamcinolone acetonide, triamcinolone alcohol is administered with a modified effector cell described herein to a subject in need thereof, wherein the steroid is administered in an amount effective to induce expansion of a population of the modified effector cell in the subject. In some instances, a steroid selected from hydrocortisone-17-butyrate, hydrocortisone-17-aceponate, hydrocortisone-17-buteprate, ciclesonide, or prednicarbate is administered with a modified effector cell described herein to a subject in need thereof, wherein the steroid is administered in an amount effective to induce expansion of a population of the modified effector cell in the subject. In some instances, a steroid selected from fluoxymesteron, mesterolone, methandrostenolone, nandrolone-undecanoate, nandrolone-cyplonate, oxandrolone, oxymetholone, nandrolone-hexyloxy phenylpropionate, testosterone, or stanozolol is administered with a modified effector cell described herein to a subject in need thereof, wherein the steroid is administered in an amount effective to induce expansion of a population of the modified effector cell in the subject. In some instances, a steroid selected from fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone or stanozolol is administered with a modified effector cell described herein to a subject in need thereof, wherein the steroid is administered in an amount effective to induce expansion of a population of the modified effector cell in the subject.

In some instances, methods described herein enable the omission of a pre-conditioning step (e.g., lymphodepletion). In such cases, modified effector cells and a steroid described herein are administered to a subject without the need of a prior administration of a chemotherapeutic agent to induce lymphopenia.

### Dosing Regimen

In some instances, expansion of a modified effector cell depends on the administrative timing of the steroid. In some embodiments, a steroid is administered simultaneously with a modified effector cell described herein. In other embodiments, a steroid is administered sequentially with a modified effector cell described herein. In some cases, a steroid is administered to a subject prior to administering a modified effector cell. In other cases, a steroid is administered to a subject after administering a modified effector cell. In some embodiments, a steroid is administered to a subject in an amount effective to induce and/or sustain expansion of a population of the modified effector cell in the subject. In some embodiments, a steroid is administered to a subject in an amount effective to not clinically interfere with engraftment and/or sustained expansion of a population of the modified effector cell(s) in the subject. In some embodiments, the amount does not adversely impact engraft and/or sustained expansion of a population of the modified effector cell in the subject.

In some cases, a steroid is administered at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 15, 16, 17, 18, 19, 20, 21, 22, 23 or 24 hours prior to administration of a modified effector cell. In some cases, a steroid is administered at least 0.5, 1, 2, 3, 4, 5, 6, 8, 10, 11, 12, 15, 18, 20, or 24 hours prior to administration of a modified effector cell. In some cases, a steroid is administered at least 0.5 hours prior to administration of a modified effector cell. In some cases, a steroid is administered at least 1 hour prior to administration of a modified effector cell. In some cases, a steroid is administered at least 1.5 hours prior to administration of a modified effector cell. In some cases, a steroid is administered at least 2 hours prior to administration of a modified effector cell. In some cases, a steroid is administered at least 3 hours prior to administration of a modified effector cell. In some cases, a steroid is administered at least 4 hours prior to administration of a modified effector cell. In some cases, a steroid is administered at least 5 hours prior to administration of a modified effector cell. In some cases, a steroid is administered at least 6 hours prior to administration of a modified effector cell. In some cases, a steroid is administered at least 7 hours prior to administration of a modified effector cell. In some cases, a steroid is administered at least 8 hours prior to administration of a modified effector cell. In some cases, a steroid is administered at least 9 hours prior to administration of a modified effector cell. In some cases, a steroid is administered at least 10 hours prior to administration of a modified effector cell. In some cases, a steroid is administered at least 12 hours prior to administration of a modified effector cell. In some cases, a steroid is administered at least 15 hours prior to administration of a modified effector cell. In some cases, a steroid is administered at least 18 hours prior to administration of a modified effector cell. In some cases, a steroid is administered at least 20 hours prior to administration of a modified effector cell. In some cases, a steroid is administered at least 24 hours prior to administration of a modified effector cell.

In some embodiments, a steroid is administered at least 2, 3, 4, 5, 6, 7, 8, 9, 10, 12, 14, 15, 21, 28 or 30 days prior to administration of a modified effector cell. In some instances, a steroid is administered at least 2 days prior to administration of a modified effector cell. In some instances, a steroid is administered at least 3 days prior to administration of a modified effector cell. In some instances, a steroid is administered at least 4 days prior to administration of a modified effector cell. In some instances, a steroid is administered at least 5 days prior to administration of a modified effector cell. In some instances, a steroid is administered at least 6 days prior to administration of a modified effector cell. In some instances, a steroid is administered at least 7 days prior to administration of a modified effector cell. In some instances, a steroid is administered at least 8 days prior to administration of a modified effector cell. In some instances, a steroid is administered at least 9 days prior to administration of a modified effector cell. In some instances, a steroid is administered at least 10 days prior to administration of a modified effector cell. In some instances, a steroid is administered at least 12 days prior to administration of a modified effector cell. In some instances, a steroid is administered at least 14 days prior to administration of a modified effector cell. In some instances, a steroid is administered at least 15 days prior to administration of a modified effector cell. In some instances, a steroid is administered at least 28 days prior to administration of a modified effector cell. In some instances, a steroid is administered at least 30 days prior to administration of a modified effector cell.

In some cases, a steroid is administered at least 0.5, 1, 2, 3, 4, 5, 6, 7, 8, 9, 10, 12, 15, 18, 20, 24, 36, 48, 60, 72, 84, 96, 108, or 120 hours after administration of the modified effector cell. In some cases, a steroid is administered at least 0.5, 1, 2, 3, 4, 5, 6, 8, 10, 12, 15, 18, 20, 24, 36, 48, 60, 72, 84, 96, 108, or 120 hours after administration of the modified effector cell. In some cases, a steroid is administered at least 0.5 hours after administration of the modified effector cell. In some cases, a steroid is administered at least 1 hour after administration of the modified effector cell. In some cases, a steroid is administered at least 1.5 hours after administration of the modified effector cell. In some cases, a steroid is administered at least 2 hours after administration of the modified effector cell. In some cases, a steroid is administered at least 3 hours after administration of the modified effector cell. In some cases, a steroid is administered at least 4 hours after administration of the modified effector cell. In some cases, a steroid is administered at least 5 hours after administration of the modified effector cell. In some cases, a steroid is administered at least 6 hours after administration of the modified effector cell. In some cases, a steroid is administered at least 7 hours after administration of the modified effector cell. In some cases, a steroid is administered at least 8 hours after administration of the modified effector cell. In some cases, a steroid is administered at least 9 hours after administration of the modified effector cell. In some cases, a steroid is administered at least 10 hours after administration of the modified effector cell. In some cases, a steroid is administered at least 12 hours after administration of the modified effector cell. In some cases, a steroid is administered at least 15 hours after administration of the modified effector cell. In some cases, a steroid is administered at least 18 hours after administration of the modified effector cell. In some cases, a steroid is administered at least 20 hours after administration of the modified effector cell. In some cases, a steroid is administered at least 24 hours after administration of the modified effector cell. In some cases, a steroid is administered at least 36 hours after administration of the modified effector cell. In some cases, a steroid is administered at least 48 hours after administration of the modified effector cell. In some cases, a steroid is administered at least 60 hours after administration of the modified effector cell. In some cases, a steroid is administered at least 72 hours after administration of the modified effector cell. In some cases, a steroid is administered at least 84 hours after administration of the modified effector cell. In some cases, a steroid is administered at least 96 hours after administration of the modified effector cell. In some cases, a steroid is administered at least 108 hours after administration of the modified effector cell. In some cases, a steroid is administered at least 120 hours after administration of the modified effector cell.

In some embodiments, a steroid is administered at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified effector cell. In some embodiments, a steroid is administered at least 2, 3, 4, 5, 6, 7, 8, 9, 10, 12, 14, 15, 28, 30, 45, 60, 90 or more days after administration of a modified effector cell. In some instances, a steroid is administered at least 2 days after administration of a modified effector cell. In some instances, a steroid is administered at least 3 days after administration of a modified effector cell. In some instances, a steroid is administered at least 4 days after administration of a modified effector cell. In some instances, a steroid is administered at least 5 days after administration of a modified effector cell. In some instances, a steroid is administered at least 6 days after administration of a modified effector cell. In some instances, a steroid is administered at least 7 days after administration of a modified effector cell. In some instances, a steroid is administered at least 8 days after administration of a modified effector cell. In some instances, a steroid is administered at least 9 days after administration of a modified effector cell. In some instances, a steroid is administered at least 10 days after administration of a modified effector cell. In some instances, a steroid is administered at least 12 days after administration of a modified effector cell. In some instances, a steroid is administered at least 14 days after administration of a modified effector cell. In some instances, a steroid is administered at least 15 days after administration of a modified effector cell. In some instances, a steroid is administered at least 28 days after administration of a modified effector cell. In some instances, a steroid is administered at least 30 days after administration of a modified effector cell. In some instances, a steroid is administered at least 45 days after administration of a modified effector cell. In some instances, a steroid is administered at least 60 days after administration of a modified effector cell. In some instances, a steroid is administered at least 90 days after administration of a modified effector cell.

In some embodiments, a steroid is administered continuously for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some instances, a steroid is administered continuously for 1, 2, 3, 4, 5, 6, 7, 8, 9, 10, 12, 15, 20, 25, 30, 45, 60, 90 or more days. In some instances, a steroid is administered continuously for 1 or more days. In some instances, a steroid is administered continuously for 2 or more days. In some instances, a steroid is administered continuously for 3 or more days. In some instances, a steroid is administered continuously for 4 or more days. In some instances, a steroid is administered continuously for 5 or more days. In some instances, a steroid is administered continuously for 6 or more days. In some instances, a steroid is administered continuously for 7 or more days. In some instances, a steroid is administered continuously for 8 or more days. In some instances, a steroid is administered continuously for 9 or more days. In some instances, a steroid is administered continuously for 10 or more days. In some instances, a steroid is administered continuously for 12 or more days. In some instances, a steroid is administered continuously for 15 or more days. In some instances, a steroid is administered continuously for 20 or more days. In some instances, a steroid is administered continuously for 25 or more days. In some instances, a steroid is administered continuously for 30 or more days. In some instances, a steroid is administered continuously for 45 or more days. In some instances, a steroid is administered continuously for 60 or more days. In some instances, a steroid is administered continuously for 90 or more days.

In some cases, a steroid is administered at predetermined time intervals for 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some cases, a steroid is administered at predetermined time intervals for 1, 2, 3, 4, 5, 6, 7, 8, 9, 10, 12, 15, 20, 25, 30, 45, 60, 90 or more days. In some cases, a steroid is administered at predetermined time intervals for 1 or more days. In some cases, a steroid is administered at predetermined time intervals for 2 or more days. In some cases, a steroid is administered at predetermined time intervals for 3 or more days. In some cases, a steroid is administered at predetermined time intervals for 4 or more days. In some cases, a steroid is administered at predetermined time intervals for 5 or more days. In some cases, a steroid is administered at predetermined time intervals for 6 or more days. In some cases, a steroid is administered at predetermined time intervals for 7 or more days. In some cases, a steroid is administered at predetermined time intervals for 8 or more days. In some cases, a steroid is administered at predetermined time intervals for 9 or more days. In some cases, a steroid is administered at predetermined time intervals for 10 or more days. In some cases, a steroid is administered at predetermined time intervals for 12 or more days. In some cases, a steroid is administered at predetermined time intervals for 15 or more days. In some cases, a steroid is administered at predetermined time intervals for 20 or more days. In some cases, a steroid is administered at predetermined time intervals for 25 or more days. In some cases, a steroid is administered at predetermined time intervals for 30 or more days. In some cases, a steroid is administered at predetermined time intervals for 45 or more days. In some cases, a steroid is administered at predetermined time intervals for 60 or more days. In some cases, a steroid is administered at predetermined time intervals for 90 or more days.

In some embodiments, a steroid is administered every 2, 5, 7, 10, 14, 15, 28 or 30 days. In some embodiments, a steroid is administered every 2 days. In some embodiments, a steroid is administered every 5 days. In some embodiments, a steroid is administered every 7 days. In some embodiments, a steroid is administered every 10 days. In some embodiments, a steroid is administered every 14 days. In some embodiments, a steroid is administered every 15 days. In some embodiments, a steroid is administered every 28 days. In some embodiments, a steroid is administered every 30 days.

In some instances, a steroid is administered once a day, two times a day, three times a day, daily, once every other day, three times a week, four times a week, five times a week, once a week, every other week, once a month, two times a month, three times a month, four times a month, five times a month, or a combination thereof.

In some cases, an amount of steroid is administered in 1 dose, 2 doses, 3 doses, 4 doses, 5 doses or more. In some cases, the amount of steroid is administered in 1 dose or more. In some cases, the amount of steroid is administered in 2 doses or more. In some cases, the amount of steroid is administered in 3 doses or more. In some cases, the amount of steroid is administered in 4 doses or more. In some cases, the amount of steroid is administered in 5 doses or more.

In some embodiments, a steroid is administered at a first amount at least 2, 3, 4, 5, 6, 7, 8, 9, 10, 12, 14, 15, 28, 30 or more days after administration of a modified effector cell and then at a second amount at least 10, 15, 20, 25, 30, 35, 40, 45, 49, 50, 55, 60, 65, 70, 75, 80, 85, 90 or more days after administration of a modified effector cell. In some instances, a steroid is administered at a first amount at least 2, 3, 4, 5, 6, 7, 8, 9, 10, 12, 14, 15, 21, 28, 30 or more days after administration of a modified effector cell for the treatment of a condition. In some cases, upon improvement or recovery from the condition in a subject, a second amount of a steroid is administered to the subject at least 15, 30, 31, 32, 33, 34, 35, 40, 45, 49, 50, 55, 60, 65, 70, 80, 90 or more days after the initial administration of a modified effector cell.

In some embodiments, the first amount of steroid is administered to the subject at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days after administration of the modified effector cell. In some embodiments, the first amount of steroid is administered to the subject at least 2, 3, 4, 5, 6, 7, 8, 9, 10, 12, 14, 15, 28, 30, 45, 60, 90 or more days after administration of the modified effector cell. In some embodiments, a steroid is administered at a first amount at least 2, 3, 4, 5, 6, 7, 8, 9, 10, 12, 14, 15, 21, 28, 30 or more days after administration of a modified effector cell. In some embodiments, a steroid is administered at a first amount at least 1, 2, 3, 4, 5, 6, 7, 8, 9, 10, 12, 13, 14, or 15 days after administration of the modified effector cell. In some embodiments, a steroid is administered at a first amount at least 14 days after administration of a modified effector cell. In some embodiments, a steroid is administered at a first amount at least 15 days after administration of a modified effector cell. In some embodiments, a steroid is administered at a first amount at least 21 days after administration of a modified effector cell. In some embodiments, a steroid is administered at a first amount at least 28 days after administration of a modified effector cell.

In some embodiments, the first amount of steroid is administered to the subject continuously for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered to the subject continuously for at least 2, 3, 4, 5, 6, 7, 8, 9, 10, 12, 14, 15, 21, 28, 30, 45, 60, 90 or more days. In some embodiments, the first amount of steroid is administered to the subject continuously for at least 1, 2, 3, 4, 5, 6, 7, 8, 9, 10, 12, 13, 14, or 15 days after administration of the modified effector cell. In some embodiments, the first amount of steroid is administered to the subject continuously for at least 14 days after administration of a modified effector cell. In some embodiments, the first amount of steroid is administered to the subject continuously for at least 15 days after administration of a modified effector cell. In some embodiments, the first amount of steroid is administered to the subject continuously for at least 21 days after administration of a modified effector cell. In some embodiments, the first amount of steroid is administered to the subject continuously for at least 28 days after administration of a modified effector cell.

In some embodiments, the first amount of steroid is administered to the subject intermittently for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some embodiments, the first amount of steroid is administered to the subject intermittently for at least 2, 3, 4, 5, 6, 7, 8, 9, 10, 12, 14, 15, 21, 28, 30, 45, 60, 90 or more days. In some embodiments, the first amount of steroid is administered to the subject intermittently for at least 1, 2, 3, 4, 5, 6, 7, 8, 9, 10, 12, 13, 14, or 15 days after administration of the modified effector cell. In some embodiments, the first amount of steroid is administered to the subject intermittently for at least 14 days after administration of a modified effector cell. In some embodiments, the first amount of steroid is administered to the subject intermittently for at least 15 days after administration of a modified effector cell. In some embodiments, the first amount of steroid is administered to the subject intermittently for at least 21 days after administration of a modified effector cell. In some embodiments, the first amount of steroid is administered to the subject intermittently for at least 28 days after administration of a modified effector cell.

In some cases, the first amount of steroid is administered to the subject at predetermined time intervals for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some cases, the first amount of steroid is administered to the subject at predetermined time intervals for at least 2, 3, 4, 5, 6, 7, 8, 9, 10, 12, 14, 15, 21, 28, 30, 45, 60, 90 or more days. In some cases, the first amount of steroid is administered to the subject at predetermined time intervals for at least 1, 2, 3, 4, 5, 6, 7, 8, 9, 10, 12, 13, 14, or 15 days after administration of the modified effector cell. In some cases, the first amount of steroid is administered to the subject at predetermined time intervals for at least 14 days after administration of the modified effector cell. In some cases, the first amount of steroid is administered to the subject at predetermined time intervals for at least 15 days after administration of the modified effector cell. In some cases, the first amount of steroid is administered to the subject at predetermined time intervals for at least 21 days after administration of the modified effector cell. In some cases, the first amount of steroid is administered to the subject at predetermined time intervals for at least 28 days after administration of the modified effector cell.

In some embodiments, the first amount of steroid is administered every 2, 5, 7, 10, 14, 15, 28 or 30 days. In some embodiments, the first amount of steroid is administered every 2 days. In some embodiments, the first amount of steroid is administered every 5 days. In some embodiments, the first amount of steroid is administered every 7 days. In some embodiments, the first amount of steroid is administered every 10 days. In some embodiments, the first amount of steroid is administered every 14 days. In some embodiments, the first amount of steroid is administered every 15 days. In some embodiments, the first amount of steroid is administered every 28 days. In some embodiments, the first amount of steroid is administered every 30 days.

In some embodiments, upon recovery or improvement of the condition, a second amount of steroid is administered to the subject. In some cases, the second amount of steroid is administered to the subject at least 2, 3, 4, 5, 6, 7, 8, 9, 10, 12, 14, 15, 28, 30, 45, 49, 50, 60, 90 or more days after administration of the modified effector cell and after administration of the first amount of steroid has been completed. In some embodiments, the second amount of steroid is administered to the subject at least 49 or more days after administration of the modified effector cell and after administration of the first amount of steroid has been completed.

In some cases, the second amount of steroid is administered to the subject continuously for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some cases, the second amount of steroid is administered to the subject continuously for at least 2, 3, 4, 5, 6, 7, 8, 9, 10, 12, 14, 15, 28, 30, 45, 60, 90 or more days. In some cases, the second amount of steroid is administered to the subject continuously for at least 10, 15, 20, 25, 30, 35, 40, 45, 49, 50, 55, 60, 65, 70, 75, 80, 85, 90 or more days.

In some cases, the second amount of steroid is administered to the subject intermittently for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some cases, the second amount of steroid is administered to the subject intermittently for at least 2, 3, 4, 5, 6, 7, 8, 9, 10, 12, 14, 15, 28, 30, 45, 60, 90 or more days. In some cases, the second amount of steroid is administered to the subject intermittently for at least 10, 15, 20, 25, 30, 35, 40, 45, 49, 50, 55, 60, 65, 70, 75, 80, 85, 90 or more days.

In some cases, the second amount of steroid is administered to the subject at predetermined time intervals for at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days. In some cases, the second amount of steroid is administered to the subject at predetermined time intervals for at least 2, 3, 4, 5, 6, 7, 8, 9, 10, 12, 14, 15, 28, 30, 45, 60, 90 or more days. In some cases, the second amount of steroid is administered to the subject at predetermined time intervals for at least 10, 15, 20, 25, 30, 35, 40, 45, 49, 50, 55, 60, 65, 70, 75, 80, 85, 90 or more days.

In some embodiments, the second amount of steroid is administered every 2, 5, 7, 10, 14, 15, 28 or 30 days. In some embodiments, the second amount of steroid is administered every 2 days. In some embodiments, the second amount of steroid is administered every 5 days. In some embodiments, the second amount of steroid is administered every 7 days. In some embodiments, the second amount of steroid is administered every 10 days. In some embodiments, the second amount of steroid is administered every 14 days. In some embodiments, the second amount of steroid is administered every 15 days. In some embodiments, the second amount of steroid is administered every 28 days. In some embodiments, the second amount of steroid is administered every 30 days.

In some instances, a first amount of steroid is administered in 1 dose, 2 doses, 3 doses, 4 doses, 5 doses or more for treating a condition.

In some cases, a second amount of steroid is administered in 1 dose, 2 doses, 3 doses, 4 doses, 5 doses or more.

In some embodiments, a first amount of a steroid is about 1 mg/kg, 1.5 mg/kg, 2 mg/kg, 2.5 mg/kg, 3 mg/kg or more. In some instances, a first amount of a steroid is about 1 mg/kg. In some instances, a first amount of a steroid is about 1.5 mg/kg. In some instances, a first amount of a steroid is about 2 mg/kg.

In some cases, a second amount of a steroid is about 0.1 mg/kg, 0.2 mg/kg, 0.3 mg/kg, 0.4 mg/kg, 0.5 mg/kg, 0.6 mg/kg, 0.7 mg/kg, 0.8 mg/kg or 0.9 mg/kg. In some cases, a second amount of a steroid is about 0.9 mg/kg. In some cases, a second amount of a steroid is about 0.8 mg/kg. In some cases, a second amount of a steroid is about 0.7 mg/kg. In some cases, a second amount of a steroid is about 0.6 mg/kg. In some cases, a second amount of a steroid is about 0.5 mg/kg. In some cases, a second amount of a steroid is about 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In some cases, the second amount of steroid is administered sequentially. In some cases, each sequential dose is reduced by 0.1 mg/kg, 0.2 mg/kg, 0.3 mg/kg, 0.4 mg/kg, 0.5 mg/kg, 0.6 mg/kg, 0.7 mg/kg, 0.8 mg/kg or 0.9 mg/kg. In some cases, each sequential dose is administered at least 2, 5, 7, 10, 12, 14, 15, 20, 21, 28, 30 or more days apart. In some cases, each sequential dose is administered 2 days apart. In some cases, each sequential dose is administered 5 days apart. In some cases, each sequential dose is administered 7 days apart. In some cases, each sequential dose is administered 10 days apart. In some cases, each sequential dose is administered 12 days apart. In some cases, each sequential dose is administered 14 days apart. In some cases, each sequential dose is administered 15 days apart. In some cases, each sequential dose is administered 20 days apart. In some cases, each sequential dose is administered 21 days apart. In some cases, each sequential dose is administered 28 days apart. In some cases, each sequential dose is administered 30 days apart.

In some embodiments, a steroid is administered at 1 mg/kg at least 2, 3, 4, 5, 6, 7, 8, 9, 10, 12, 14, 15, 28, 30 or more days after administration of a modified effector cell and then at 0.5 mg/kg at least 30, 35, 40, 45, 49, 50, 55, 60, 65, 70, 80, 90 or more days after the initial administration of a modified effector cell. In some embodiments, a steroid is administered at 1 mg/kg at least 15 days after administration of a modified effector cell and then at 0.5 mg/kg at least 49 days after the initial administration of a modified effector cell. In some embodiments, a steroid is administered at 1 mg/kg on day 14 post administration of a modified effector cell and then at 0.5 mg/kg on day 48 post administration of the modified effector cell.

In some embodiments, a steroid is administered upon manifestation of at least one symptom of cytokine-associated toxicity. In some cases, a cytokine-associated toxicity comprises cytokine release syndrome (CRS), encephalopathy or B-cell aplasia. In some cases, a cytokine-associated toxicity induces a cytokine storm.

In some cases, the first amount of steroid is administered to the subject for treating a condition. In some embodiments, the condition is graft versus host disease (GVHD). In some embodiments, the condition is cytokine release syndrome (CRS).

In some instances, a steroid is administered in an amount that is less than an amount of steroid needed for a reduction in at least one symptom of cytokine-associated toxicity.

### Cytokines

In some embodiments, one or more methods described herein further comprise administration of a cytokine. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25. CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily: XCL1 and XCL2: and the CX3C subfamily CX3CL1.

Interferons (IFNs) comprise interferon type I (e.g. IFN-α, IFN-β, IFN-ε, IFN-κ, and IFN-ω), interferon type II (e.g. IFN-γ), and interferon type III. In some embodiments, IFN-α is further classified into about 13 subtypes including IFNA1, IFNA2, IFNA4, IFNA5, IFNA6, IFNA7, IFNA8, IFNA10, IFNA13, IFNA14, IFNA16, IFNA17, and IFNA21.

Interleukins are expressed by leukocytes or white blood cells and they promote the development and differentiation of T and B lymphocytes and hematopoietic cells. Exemplary interleukines include IL-1, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36.

In some embodiments, an interleukin comprises mbIL-15. In some embodiments, a mbIL-15 is a membrane-bound chimeric IL-15 which can be co-expressed with a modified effector cell described herein. In some embodiments, the mbIL-15 comprises a full-length IL-15 (e.g., a native IL-15 polypeptide) or fragment or variant thereof, fused in frame with a full length IL-15Rα, functional fragment or variant thereof. In some cases, the IL-15 is indirectly linked to the IL-15Rα through a linker. In some instances, the mbIL-15 is as described in Hurton et al., “Tethered IL-15 augments antitumor activity and promotes a stem-cell memory subset in tumor-specific T cells,” PNAS 2016.

Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In some embodiments, the cytokine is a membrane-bound cytokine, which is co-expressed with a chimeric antigen receptor described herein.

In some embodiments, one or more methods described herein further comprise administration of a cytokine. In some instances, the cytokine comprises a chemokine, an interferon, an interleukin, a colony-stimulating factor or a tumor necrosis factor. In some instances, one or more methods described herein further comprise administration of a cytokine selected from a chemokine, an interferon, an interleukin, a colony-stimulating factor or a tumor necrosis factor. In some instances, one or more methods described herein further comprise administration of a cytokine selected from IL-2, IL-7, IL-12, IL-15, IL-21, IFNγ or TNF-α.

In some instances, the cytokine is administered simultaneously with the steroid or the modified effector cell. In some cases, the cytokine is administered simultaneously with the steroid and the modified effector cell.

In some cases, the cytokine is administered sequentially with the steroid and/or the modified effector cell.

### Additional Therapeutic Agents

In some embodiments, one or more additional therapeutic agents are administered with a method described herein. In some instances, the one or more additional therapeutic agents comprise chemotherapeutic agents. Exemplary chemotherapeutic agents include:
Alkylating agents such as cyclophosphamide, mechlorethamine, chlorambucil, melphalan, or nitrosoureas;
Anthracyclines such as daunorubicin, doxorubicin, epirubicin, idarubicin, mitoxantrone, or valrubicin;
Taxanes such as paclitaxel, docetaxel, abraxane or taxotere;
Epothilones;
Histone deacetylase inhibitors such as vorinostat or romidepsin;
Topoisomerase inhibitors such as irinotecan, topotecan, etoposide, teniposide, or tafluposide;
Kinase inhibitors such as bortezomib, erlotinib, gefitinib, imatinib, vemurafenib, or vismodegib;
Nucleotide analogs and precursor analogs such as azacitidine, azathioprine, capecitabine, cladribine, clofarabine, cytarabine, decitabine, doxifluridine, fludarabine, fluorouracil, floxuridine, gemcitabine, hydroxyurea, mercaptopurine, methotrexate, nelarabine, pemetrexed, pralatrexate, or tioguanine;
Antimicrobials such as bleomycine or actinomycin;
Platinum-based agents such as carboplatin, cisplatin, or oxaliplatin;
Retinoids such as tretinoin, alitretinoin, or vexarotene; or
Vinca alkaloid and derivatives such as vinblastine, vincristine, vindesine, or vinorelbine.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine).

In some embodiments, the additional therapeutic agent comprises the R-CHOP regiment (rituximab, cyclophosphamide, doxorubicin, vincristine, and prednisone).

In some embodiments, the additional therapeutic agent comprises the FCR regimen (FCR (fludarabine, cyclophosphamide, rituximab).

In some embodiments, the additional therapeutic agent comprises the FCMR regimen (fludarabine, cyclophosphamide, mitoxantrone, rituximab).

In some embodiments, the additional therapeutic agent comprises the FMR regimen (fludarabine, mitoxantrone, rituximab).

In some embodiments, the additional therapeutic agent comprises the PCR regimen (pentostatin, cyclophosphamide, rituximab).

In some embodiments, the additional therapeutic agent comprises the PEPC regimen (prednisone, etoposide, procarbazine, cyclophosphamide).

In some embodiments, the additional therapeutic agent comprises a regimen of cyclophosphamide and fludarabine.

In some instances, the additional therapeutic agent is administered simultaneously with the steroid, the modified effector cell, the cytokine or a combination thereof. In some cases, the additional therapeutic agent is administered simultaneously with the steroid and the modified effector cell. In some cases, the additional therapeutic agent is administered sequentially with the steroid, the modified effector cell and/or the cytokine.

In some instances, the additional therapeutic agent is administered as a low dose regimen. In some cases, the low dose regimen comprises a regimen of cyclophosphamide and fludarabine. In some cases, the low dose regimen comprises a dose of about 250 mg/m² for cyclophosphamide and a dose of about 25 mg/m² for fludarabine. In some cases, the dosing regimen comprises about 1, 2, 3 or more days.

In other instances, the additional therapeutic agent is administered as a high dose regimen. In some cases, the high dose regimen comprises a regimen of cyclophosphamide and fludarabine. In some cases, the high dose regimen comprises a dose of about 60 mg/m² for cyclophosphamide for about 1, 2 or more days and a dose of about 25 mg/m² for fludarabine for about 1, 2, 3, 4, 5 or more days.

In some cases, the additional therapeutic agent comprises a dose regimen comprising about 250 mg/m² of cyclophosphamide and about 25 mg/m² of fludarabine for about 1, 2, 3 or more days. In some cases, the fludarabine is administered for about 3 days.

In some cases, the additional therapeutic agent comprises a dose regimen comprising about 300 mg/m² of cyclophosphamide for about 1, 2, 3, 4, 5, 6 or more days. In some cases, the cyclophosphamide is administered for about 6 days.

In some cases, the additional therapeutic agent comprises a dose regimen comprising about 500 mg/m² of cyclophosphamide and about 30 mg/m² of fludarabine for about 1, 2, 3 or more days. In some cases, the fludarabine is administered for about 3 days.

In some instances, administration of any one or more chemotherapeutic agents described above induces lymphopenia.

### Indications

In some embodiments, disclosed herein are methods of administering a modified effector cell and a steroid to a subject having a cancer. In some cases, the cancer is a cancer associated with an overexpression of CD19, CD20, CD33, CD44, BCMA, CD123, EGFRvIII, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MUC-16, MAGE-A1, h5T4, PSMA, TAG-72 or VEGF-R2. In some cases, the cancer is a cancer associated with an overexpression of CD19, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MUC-16, MAGE-A1, h5T4, PSMA, TAG-72, EGFRvIII, CD123 and VEGF-R2.

In some embodiments, disclosed herein are methods of administering a modified effector cell and a steroid to a subject having a cancer associated with an overexpression of CD19. In some embodiments, disclosed herein are methods of administering a modified effector cell and a steroid to a subject having a cancer associated with an overexpression of CD33. In some embodiments, disclosed herein are methods of administering a modified effector cell and a steroid to a subject having a cancer associated with an overexpression of CD44, BCMA, CD123, EGFRvIII, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MAGE-A1, h5T4, PSMA, TAG-72 or VEGF-R2. In some cases, the cancer is a metastatic cancer. In other cases, the cancer is a relapsed or refractory cancer.

In some cases, a cancer is a solid tumor or a hematologic malignancy. In some instances, the cancer is a solid tumor. In other instances, the cancer is a hematologic malignancy. In some cases, the cancer is a metastatic cancer. In some cases, the cancer is a relapsed or refractory cancer.

In some instances, the cancer is a solid tumor. Exemplary solid tumors include, but are not limited to, anal cancer, appendix cancer, bile duct cancer (i.e., cholangiocarcinoma); bladder cancer; brain tumor; breast cancer, cervical cancer; colon cancer, cancer of Unknown Primary (CUP); esophageal cancer; eye cancer, fallopian tube cancer; gastroenterological cancer; kidney cancer; liver cancer; lung cancer, medulloblastoma; melanoma; oral cancer, ovarian cancer, pancreatic cancer; parathyroid disease; penile cancer; pituitary tumor; prostate cancer, rectal cancer; skin cancer, stomach cancer; testicular cancer, throat cancer, thyroid cancer; uterine cancer; vaginal cancer, or vulvar cancer.

In some instances, disclosed herein are methods of administering to a subject having a solid tumor a modified effector cell and a steroid in an amount effective to induce expansion of the population to the modified effector cell in the subject. In some cases, disclosed herein are methods of administering to a subject having a solid tumor selected from anal cancer, appendix cancer; bile duct cancer (i.e., cholangiocarcinoma); bladder cancer; brain tumor; breast cancer, cervical cancer; colon cancer, cancer of Unknown Primary (CUP); esophageal cancer, eye cancer; fallopian tube cancer, gastroenterological cancer; kidney cancer; liver cancer; lung cancer; medulloblastoma; melanoma; oral cancer, ovarian cancer; pancreatic cancer; parathyroid disease; penile cancer; pituitary tumor; prostate cancer; rectal cancer; skin cancer; stomach cancer; testicular cancer, throat cancer, thyroid cancer; uterine cancer; vaginal cancer, or vulvar cancer a modified effector cell and a steroid in an amount effective to induce expansion of the population to the modified effector cell in the subject. In some cases, the solid tumor is a metastatic solid tumor. In other cases, the solid tumor is a relapsed or refractory tumor.

In some instances, the cancer is a hematologic malignancy. In some cases, a hematologic malignancy comprises a lymphoma, a leukemia, a myeloma, or a B-cell malignancy. In some cases, a hematologic malignancy comprises a lymphoma, a leukemia or a myeloma. In some instances, exemplary hematologic malignancies include chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic malignancy comprises a myeloid leukemia. In some embodiments, the hematologic malignancy comprises acute myeloid leukemia (AML) or chronic myeloid leukemia (CML).

In some instances, disclosed herein are methods of administering to a subject having a hematologic malignancy a modified effector cell and a steroid in an amount effective to induce expansion of the population to the modified effector cell in the subject. In some instances, disclosed herein are methods of administering to a subject having a lymphoma, a leukemia, a myeloma, or a B-cell malignancy a modified effector cell and a steroid in an amount effective to induce expansion of the population to the modified effector cell in the subject. In some instances, disclosed herein are methods of administering to a subject having a hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis a modified effector cell and a steroid in an amount effective to induce expansion of the population to the modified effector cell in the subject. In some instances, disclosed herein are methods of administering to a subject having a hematologic malignancy selected from AML or CML a modified effector cell and a steroid in an amount effective to induce expansion of the population to the modified effector cell in the subject. In some instances, disclosed herein are methods of administering to a subject having large B cell lymphoma a modified effector cell and a steroid in an amount effective to induce expansion of the population to the modified effector cell in the subject. In some instances, disclosed herein are methods of administering to a subject having CLL a modified effector cell and a steroid in an amount effective to induce expansion of the population to the modified effector cell in the subject. In some instances, disclosed herein are methods of administering to a subject having a follicular lymphoma a modified effector cell and a steroid in an amount effective to induce expansion of the population to the modified effector cell in the subject. In some instances, disclosed herein are methods of administering to a subject having ALL a modified effector cell and a steroid in an amount effective to induce expansion of the population to the modified effector cell in the subject. In some instances, disclosed herein are methods of administering to a subject having AML a modified effector cell and a steroid in an amount effective to induce expansion of the population to the modified effector cell in the subject. In some instances, disclosed herein are methods of administering to a subject having CML a modified effector cell and a steroid in an amount effective to induce expansion of the population to the modified effector cell in the subject. In some instances, the hematologic malignancy is a metastatic hematologic malignancy. In other instances, the hematologic malignancy is a relapsed or refractory hematologic malignancy.

### Viral Based Delivery Systems

The present invention also provides delivery systems, such as viral-based systems, in which a nucleic acid described herein is inserted. Representative viral expression vectors include, but are not limited to, adeno-associated viral vectors, adenovirus-based vectors (e.g., the adenovirus-based Per.C6 system available from Crucell, Inc. (Leiden, The Netherlands)), lentivirus-based vectors (e.g., the lentiviral-based pLPI from Life Technologies (Carlsbad, Calif.)), retroviral vectors (e.g., the pFB-ERV plus pCFB-EGSH), and herpes virus-based vectors. In an embodiment, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In an additional embodiment, the viral vector is an adeno-associated viral vector. In a further embodiment, the viral vector is a retroviral vector. In general, and in embodiments, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFNIOA (ACT) FLEXI™ from Promega (Madison, Wis.). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

Another example of a suitable promoter is human elongation growth factor 1 alpha 1 (hEF1a1). In embodiments, the vector construct comprising the CARs and/or TCRs of the invention comprises hEF1a1 functional variants.

However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR or TCR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neomycin resistance gene (neo) and ampicillin resistance gene and the like. In some embodiments, a truncated epidermal growth factor receptor (HER1t) tag may be used as a selectable marker gene.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479: 79-82 (2000)). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vectors comprise a hEF1a1 promoter to drive expression of transgenes, a bovine growth hormone polyA sequence to enhance transcription, a woodchuck hepatitis virus posttranscriptional regulatory element (WPRE), as well as LTR sequences derived from the pFUGW plasmid.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (Molecular Cloning: A Laboratory Manual, Cold Spring Harbor Laboratory, New York (2001)). In embodiments, a method for the introduction of a polynucleotide into a host cell is calcium phosphate transfection or polyethylenimine (PEI) Transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Non-Viral Based Delivery Systems

In some instances, CARs and/or TCRs described herein can also be introduced into T cells using non-viral based delivery systems, such as the “Sleeping Beauty (SB) Transposon System,” which refers a synthetic DNA transposon system to introduce DNA sequences into the chromosomes of vertebrates. The system is described, for example, in U.S. Pat. No. 6,489,458. The Sleeping Beauty transposon system is composed of a Sleeping Beauty (SB) transposase and a SB transposon. DNA transposons translocate from one DNA site to another in a simple, cut-and-paste manner. Transposition is a precise process in which a defined DNA segment is excised from one DNA molecule and moved to another site in the same or different DNA molecule or genome. As do other Tcl/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. SB transposons envisaged to be used as non-viral vectors for introduction of genes into genomes of vertebrate animals and for gene therapy. Briefly, the Sleeping Beauty (SB) system (Hackett et al., Mol Ther 18:674-83, (2010)) was adapted to genetically modify the T cells (Cooper et al., Blood 105:1622-31, (2005)). This involved two steps: (i) the electro-transfer of DNA plasmids expressing a SB transposon [i.e., chimeric antigen receptor (CAR) to redirect T-cell specificity (Jin et al., Gene Ther 18:849-56, (2011); Kebriaei et al., Hum Gene Ther 23:444-50, (2012)) and SB transposase and (ii) the propagation and expansion of T cells stably expressing integrants on designer artificial antigen-presenting cells (AaPC) derived from the K562 cell line (also known as AaPCs (Activating and Propagating Cells). In one embodiment, the SB transposon system includes coding sequence encoding tdIL-15, an IL-21 and/or a chimeric antigen receptor. Such systems are described for example in Singh et al., Cancer Res (8):68 (2008). Apr. 15, 2008 and Maiti et al., J Immunother. 36(2): 112-123 (2013), incorporated herein by reference in their entireties.

In some embodiments, a modified effector cell (e.g., a CAR effector cell or a TCR effector cell) described herein and mbIL-15 is encoded in a transposon DNA plasmid vector, and the SB transposase is encoded in a separate vector. In embodiments, the CD19 CAR is encoded in a transposon DNA plasmid vector, mbIL-15 is encoded in a second transposon DNA plasmid vector, and the SB transposase is encoded in a third DNA plasmid vector. In some embodiments, the mbIL-15 is encoded with a truncated epidermal growth factor receptor tag.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In embodiments, a modified effector cell described herein and other genetic elements are delivered to a cell using the SB11 transposon system, the SB100× transposon system, the SB110 transposon system, the piggyBac transposon system (see, e.g., Wilson et al, “PiggyBac Transposon-mediated Gene Transfer in Human Cells,” Molecular Therapy 15:139-145 (2007), incorporated herein by reference in its entirety) and/or the piggyBac transposon system (see, e.g., Mitra et al., “Functional characterization of piggyBac from the bat Myotis lucifugus unveils an active mammalian DNA transposon,” Proc. Natl. Acad. Sci USA 110:234-239 (2013). Additional transposases and transposon systems are provided in U.S. Pat. Nos. 7,148,203; 8,227,432; U.S. Patent Publn. No. 2011/0117072; Mates et al., Nat Genet, 41(6):753-61 (2009). doi: 10.1038/ng.343. Epub 2009 May 3, Gene Ther., 18(9):849-56 (2011). doi: 10.1038/gt.2011.40. Epub 2011 Mar. 31 and in Ivics et al., Cell. 91(4):501-10, (1997), each of which is incorporated herein by reference in their entirety.

### T Cell Sources

In certain aspects, the embodiments described herein include methods of making and/or expanding the antigen-specific redirected immune effector cells (e.g., T-cells, NK-cell or NK T-cells) that comprises transfecting the cells with an expression vector containing a DNA (or RNA) construct encoding the CAR, then, optionally, stimulating the cells with feeder cells, recombinant antigen, or an antibody to the receptor to cause the cells to proliferate. In certain aspects, the cell (or cell population) engineered to express a CAR or TCR is a stem cell, iPS cell, immune effector cell or a precursor of these cells.

Sources of immune effector cells can include both allogeneic and autologous sources. In some cases immune effector cells may be differentiated from stem cells or induced pluripotent stem cells (iPSCs). Thus, cell for engineering according to the embodiments can be isolated from umbilical cord blood, peripheral blood, human embryonic stem cells, Pan-T cells or iPSCs. For example, allogeneic T cells can be modified to include a chimeric antigen receptor (and optionally, to lack functional TCR). In some aspects, the immune effector cells are primary human T cells such as T cells derived from human peripheral blood mononuclear cells (PBMC). PBMCs can be collected from the peripheral blood or after stimulation with G-CSF (Granulocyte colony stimulating factor) from the bone marrow, or umbilical cord blood. In some aspects, T cells can be purified from PBMCs prior to gene transfer. Following transfection or transduction (e.g., with a CAR expression construct), the cells may be immediately infused or may be cryopreserved. In some aspects, following transfection, the cells may be maintained in a cytokine bath which can include IL-2 and/or IL-21. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the chimeric antigen receptor is expanded ex vivo. The clone selected for expansion demonstrates the capacity to specifically recognize and lyse antigen-expressing target cells. The recombinant T cells may be expanded by stimulation with IL-2, or other cytokines that bind the common gamma-chain (e.g., IL-7, IL-12, IL-15, IL-21, and others). The recombinant T cells may be expanded by stimulation with artificial antigen presenting cells. The recombinant T cells may be expanded on artificial antigen presenting cell or with an antibody, such as OKT3, which cross links CD3 on the T cell surface. Subsets of the recombinant T cells may be further selected with the use of magnetic bead based isolation methods and/or fluorescence activated cell sorting technology and further cultured with the AaPCs. In a further aspect, the genetically modified cells may be cryopreserved.

T cells can also be obtained from a number of sources, including peripheral blood, bone marrow, lymph node tissue, cord blood, thymus tissue, tissue from a site of infection, ascites, pleural effusion, spleen tissue, and tumor (tumor-infiltrating lymphocytes). In certain embodiments of the present invention, any number of T cell lines available in the art, may be used. In certain embodiments of the present invention, T cells can be obtained from a unit of blood collected from a subject using any number of techniques known to the skilled artisan, such as Ficoll® separation. In embodiments, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Cα²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL® gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, (1991); Henderson et al., Immun 73:316-321, (1991); Bierer et al., Curr. Opin. Immun 5:763-773, (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, (1998); Haanen et al., J. Exp. Med. 190(9):13191328, (1999); Garland et al., J. Immunol Meth. 227(1-2):53-63, (1999)).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In embodiments, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one ratio being at least 1:1 particles per T cell. In one embodiment, a ratio of particles to cells of 1:1 or less is used. In one particular embodiment, the particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1, or MACS® MicroBeads from Miltenyi Biotec) are combined in a buffer, for example, PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-.gamma., IL-4, IL-7, GM-CSF, IL-10, IL-12, IL-15, TGFbeta, and TNF-alpha or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, alpha-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some cases, immune effector cells of the embodiments (e.g., T-cells) are co-cultured with activating and propagating cells (AaPCs), to aid in cell expansion. AaPCs can also be referred to as artificial Antigen Presenting cells (aAPCs). For example, antigen presenting cells (APCs) are useful in preparing therapeutic compositions and cell therapy products of the embodiments. In one aspect, the AaPCs may be transgenic K562 cells. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009, each of which is incorporated by reference. In yet a further aspect of the embodiments, culturing the transgenic CAR cells comprises culturing the transgenic CAR cells in the presence of dendritic cells or activating and propagating cells (AaPCs) that stimulate expansion of the CAR-expressing immune effector cells. In still further aspects, the AaPCs comprise a CAR-binding antibody or fragment thereof expressed on the surface of the AaPCs. The AaPCs may comprise additional molecules that activate or co-stimulate T-cells in some cases. The additional molecules may, in some cases, comprise membrane-bound Cγ cytokines. In yet still further aspects, the AaPCs are inactivated or irradiated, or have been tested for and confirmed to be free of infectious material. In still further aspects, culturing the transgenic CAR cells in the presence of AaPCs comprises culturing the transgenic CAR cells in a medium comprising soluble cytokines, such as IL-15, IL-21 and/or IL-2. The cells may be cultured at a ratio of about 10:1 to about 1:10; about 3:1 to about 1:5; about 1:1 to about 1:3 (immune effector cells to AaPCs); or any range derivable therein. For example, the co-culture of T cells and AaPCs can be at a ratio of about 1:1, about 1:2 or about 1:3.

In one aspect, the AaPCs may express CD137L. In other aspects, the AaPCs may further express CD19, CD64, CD86, or mbIL-15. In certain aspects, the AaPCs may express at least one anti-CD3 antibody clone, such as, for example, OKT3 and/or UCHT1. In one aspect, the AaPCs may be inactivated (e.g., irradiated). In one aspect, the AaPCs may have been tested for and confirmed to be free of infectious material. Methods for producing such AaPCs are known in the art. In one aspect, culturing the CAR-modified T cell population with AaPCs may comprise culturing the cells at a ratio of about 10:1 to about 1:10; about 3:1 to about 1:5; about 1:1 to about 1:3 (T cells to AaPCs); or any range derivable therein. For example, the co-culture of T cells and AaPCs can be at a ratio of about 1:1, about 1:2 or about 1:3. In one aspect, the culturing step may further comprise culturing with an aminobisphosphonate (e.g., zoledronic acid).

In a further aspect, the population of CAR-T cells is cultured and/or stimulated for no more than 7, 14, 21, 28, 35 42 days, 49, 56, 63 or 70 days. In some embodiments, the population of CAR-T cells is cultured and/or stimulated for at least 0, 2, 4, 6, 8, 10, 12, 14, 16, 18, 20, 22, 24, 26, 28, 30 or more days. In some embodiments, the population of CAR-T cells is cultured and/or stimulated for at least 5, 10, 15, 20, 25, 30, 35, 40, 45, 50, 55, 60 or more days. In some embodiments, the population of CAR-T cells is cultured and/or stimulated for at least 7, 14, 21, 28, 35, 42, 49, 56, 63 or more days. In other embodiments, a stimulation includes the co-culture of the CAR-T cells with AaPCs to promote the growth of CAR positive T cells. In another aspect, the population of transgenic CAR cells is stimulated for not more than: 1× stimulation, 2× stimulation, 3× stimulation, 4× stimulation, 5× stimulation, 5× stimulation, 6× stimulation, 7× stimulation, 8× stimulation, 9× stimulation or 10× stimulation. In some instances, the transgenic cells are not cultured ex vivo in the presence of AaPCs. In some specific instances, the method of the embodiment further comprises enriching the cell population for CAR-expressing immune effector cells (e.g., T-cells) after the transfection and/or culturing step. The enriching may comprise fluorescence-activated cell sorting (FACS) and sorting for CAR-expressing cells. In a further aspect, the sorting for CAR-expressing cells comprises use of a CAR-binding antibody. The enriching may also comprise depletion of CD56+ cells. In yet still a further aspect of the embodiment, the method further comprises cryopreserving a sample of the population of transgenic CAR cells.

In some cases, AaPCs are incubated with a peptide of an optimal length that allows for direct binding of the peptide to the MHC molecule without additional processing. Alternatively, the cells can express an antigen of interest (i.e., in the case of MHC-independent antigen recognition). Furthermore, in some cases, APCs can express an antibody that binds to either a specific CAR polypeptide or to CAR polypeptides in general (e.g., a universal activating and propagating cell (uAPC). Such methods are disclosed in WO/2014/190273, which is incorporated herein by reference. In addition to peptide-MHC molecules or antigens of interest, the AaPC systems may also comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD70 and B7.1 (B7.1 was previously known as B7 and also known as CD80), which among other things, bind to CD28 and/or CTLA-4 molecules on the surface of T cells, thereby affecting, for example, T-cell expansion, Th1 differentiation, short-term T-cell survival, and cytokine secretion such as interleukin (IL)-2. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001, incorporated herein by reference.

Cells selected to become AaPCs, preferably have deficiencies in intracellular antigen-processing, intracellular peptide trafficking, and/or intracellular MHC Class I or Class II molecule-peptide loading, or are poikilothermic (i.e., less sensitive to temperature challenge than mammalian cell lines), or possess both deficiencies and poikilothermic properties. Preferably, cells selected to become AaPCs also lack the ability to express at least one endogenous counterpart (e.g., endogenous MHC Class I or Class II molecule and/or endogenous assisting molecules as described above) to the exogenous MHC Class I or Class II molecule and assisting molecule components that are introduced into the cells. Furthermore, AaPCs preferably retain the deficiencies and poikilothermic properties that were possessed by the cells prior to their modification to generate the AaPCs. Exemplary AaPCs either constitute or are derived from a transporter associated with antigen processing (TAP)-deficient cell line, such as an insect cell line. An exemplary poikilothermic insect cells line is a Drosophila cell line, such as a Schneider 2 cell line (see, e.g., Schneider 1972 Illustrative methods for the preparation, growth, and culture of Schneider 2 cells, are provided in U.S. Pat. Nos. 6,225,042, 6,355,479, and 6,362,001.

In one embodiment, AaPCs are also subjected to a freeze-thaw cycle. In an exemplary freeze-thaw cycle, the AaPCs may be frozen by contacting a suitable receptacle containing the AaPCs with an appropriate amount of liquid nitrogen, solid carbon dioxide (i.e., dry ice), or similar low-temperature material, such that freezing occurs rapidly. The frozen APCs are then thawed, either by removal of the AaPCs from the low-temperature material and exposure to ambient room temperature conditions, or by a facilitated thawing process in which a lukewarm water bath or warm hand is employed to facilitate a shorter thawing time. Additionally, AaPCs may be frozen and stored for an extended period of time prior to thawing. Frozen AaPCs may also be thawed and then lyophilized before further use. Preferably, preservatives that might detrimentally impact the freeze-thaw procedures, such as dimethyl sulfoxide (DMSO), polyethylene glycols (PEGs), and other preservatives, are absent from media containing AaPCs that undergo the freeze-thaw cycle, or are essentially removed, such as by transfer of AaPCs to media that is essentially devoid of such preservatives.

In further embodiments, xenogenic nucleic acid and nucleic acid endogenous to the AaPCs, may be inactivated by crosslinking, so that essentially no cell growth, replication or expression of nucleic acid occurs after the inactivation. In one embodiment, AaPCs are inactivated at a point subsequent to the expression of exogenous MHC and assisting molecules, presentation of such molecules on the surface of the AaPCs, and loading of presented MHC molecules with selected peptide or peptides. Accordingly, such inactivated and selected peptide loaded AaPCs, while rendered essentially incapable of proliferating or replicating, retain selected peptide presentation function. Preferably, the crosslinking also yields AaPCs that are essentially free of contaminating microorganisms, such as bacteria and viruses, without substantially decreasing the antigen-presenting cell function of the AaPCs. Thus crosslinking maintains the important AaPC functions of while helping to alleviate concerns about safety of a cell therapy product developed using the AaPCs. For methods related to crosslinking and AaPCs, see for example, U.S. Patent Application Publication No. 20090017000, which is incorporated herein by reference.

In certain embodiments there are further provided an engineered antigen presenting cell (APC). Such cells may be used, for example, as described above, to propagate immune effector cells ex vivo. In further aspects, engineered APCs may, themselves be administered to a patient and thereby stimulate expansion of immune effector cells in vivo. Engineered APCs of the embodiments may, themselves, be used as a therapeutic agent. In other embodiments, the engineered APCs can used as a therapeutic agent that can stimulate activation of endogenous immune effector cells specific for a target antigen and/or to increase the activity or persistence of adoptively transferred immune effector cells specific to a target antigen.

As used herein the term “engineered APC” refers to cell(s) that comprises at least a first transgene, wherein the first transgene encodes a HLA. Such engineered APCs may further comprise a second transgene for expression of an antigen, such that the antigen is presented at the surface on the APC in complex with the HLA. In some aspects, the engineered APC can be a cell type that presented antigens (e.g., a dendritic cell). In further aspects, engineered APC can be produced from a cell type that does not normally present antigens, such a T-cell or T-cell progenitor (referred to as “T-APC”). Thus, in some aspects, an engineered APC of the embodiments comprises a first transgene encoding a target antigen and a second transgene encoding a human leukocyte antigen (HLA), such that the HLA is expressed on the surface of the engineered APC in complex with an epitope of the target antigen. In certain specific aspects, the HLA expressed in the engineered APC is HLA-A2.

In some aspects, an engineered APC of the embodiments may further comprise at least a third transgene encoding co-stimulatory molecule. The co-stimulatory molecule may be a co-stimulatory cytokine that may be a membrane-bound Cγ cytokine. In certain aspects, the co-stimulatory cytokine is IL-15, such as membrane-bound IL-15. In some further aspects, an engineered APC may comprise an edited (or deleted) gene. For example, an inhibitory gene, such as PD-1, LIM-3, CTLA-4 or a TCR, can be edited to reduce or eliminate expression of the gene. An engineered APC of the embodiments may further comprise a transgene encoding any target antigen of interest. For example, the target antigen can be an infectious disease antigen or a tumor-associated antigen (TAA).

In one embodiment of the present invention, the T cells described herein are modified at a point-of-care site. In some cases, the point-of-care site is at a hospital or at a facility (e.g., a medical facility) near a subject. In some cases, the T cells are modified by engineering/introducing a chimeric receptor into the T cells and then rapidly infused into a subject. In some cases, the modified T cells do not undergo a propagation and activation step. In some cases, the modified T cells do not undergo an incubation step. In other cases, the T cells are modified by engineering/introducing a chimeric receptor and a cytokine into the T cells and then rapidly infused into a subject. In one case, the cytokine can be mbIL-15.

### Pharmaceutical Compositions and Dosage Forms

In some embodiments, disclosed herein are modified effector cell compositions for administration in a subject. In some instances, the modified effector cell composition further comprises a steroid, and optionally a cytokine and/or an additional therapeutic agent. In some instances, also included herein are vectors encoding a chimeric antigen receptor for modification of an effector cell.

In some instances, pharmaceutical compositions of a modified effector cell or a vector encoding a chimeric antigen receptor are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer, crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and An anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar, monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include CAR-T cells (e.g., CD19 CAR-T cells) and a steroid described herein, and optionally in addition with cytokines and/or chemotherapeutic agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

As used herein, ranges and amounts can be expressed as “about” a particular value or range. About also includes the exact amount. Hence “about 5 ML” means “about 5 ML” and also “5 ML.” Generally, the term “about” includes an amount that would be expected to be within experimental error.

By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” It is to be understood, however, that nucleic acids and proteins may be formulated with diluents or adjuvants and still for practical purposes be isolated. For example, nucleic acids typically are mixed with an acceptable carrier or diluent when used for introduction into cells.

“Polynucleotide” or “oligonucleotide” as used herein refers to a polymeric form of nucleotides of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs.

“Polypeptide” is used interchangeably with the terms “polypeptides” and “protein(s)”, and refers to a polymer of amino acid residues. A “mature protein” is a protein which is full-length and which, optionally, includes glycosylation or other modifications typical for the protein in a given cellular environment.

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology.

The terms “identical” or “sequence identity” in the context of two nucleic acid sequences or amino acid sequences of polypeptides refers to the residues in the two sequences which are the same when aligned for maximum correspondence over a specified comparison window.

In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

Proteins disclosed herein (including functional portions and functional variants thereof) may comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

“Transposon” or “transposable element” (TE) is a vector DNA sequence that can change its position within the genome, sometimes creating or reversing mutations and altering the cell's genome size. Transposition often results in duplication of the TE. Class I TEs are copied in two stages: first they are transcribed from DNA to RNA, and the RNA produced is then reverse transcribed to DNA. This copied DNA is then inserted at a new position into the genome. The reverse transcription step is catalyzed by a reverse transcriptase, which may be encoded by the TE itself. The characteristics of retrotransposons are similar to retroviruses, such as HIV. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons may be identified by short direct repeats which may be created by the staggered cut in the target DNA and filling in by DNA polymerase, followed by a series of inverted repeats important for the TE excision by transposase. Cut-and-paste TEs may be duplicated if their transposition takes place during S phase of the cell cycle when a donor site has already been replicated, but a target site has not yet been replicated. Transposition can be classified as either “autonomous” or “non-autonomous” in both Class I and Class II TEs. Autonomous TEs can move by themselves while non-autonomous TEs require the presence of another TE to move. This is often because non-autonomous TEs lack transposase (for class II) or reverse transcriptase (for class I).

“Transposase” refers an enzyme that binds to the end of a transposon and catalyzes the movement of the transposon to another part of the genome by a cut and paste mechanism or a replicative transposition mechanism.

“Sleeping Beauty (SB) Transposon System” refers a synthetic DNA transposon system for to introducing DNA sequences into the chromosomes of vertebrates. The system is described for example in U.S. Pat. Nos. 6,489,458 and 8,227,432.

The nucleic acid sequences and vectors disclosed or contemplated herein may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

“Promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter.

The term “promoter activity” refers to the extent of expression of nucleotide sequence that is operably linked to the promoter whose activity is being measured. Promoter activity may be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters are alcohol-regulated promoters, tetracycline-regulated promoters, steroid-regulated promoters, metal-regulated promoters, pathogenesis-regulated promoters, temperature-regulated promoters and light-regulated promoters. In one embodiment, the inducible promoter is part of a genetic switch.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences.

Vector also can comprise a “selectable marker gene.” The term “selectable marker gene,” as used herein, refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150:1 (1981); Santerre et al., Gene, 30: 147 (1984); Kent et al., Science, 237: 901-903 (1987); Wigler et al., Cell, 11: 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

The terms “fragment of an antibody,” “antibody fragment,” “functional fragment of an antibody,” and “antigen-binding portion” are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

The term “functional portion,” when used in reference to a CAR, refers to any part or fragment of the CAR of the invention, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The term “functional variant,” as used herein, refers to a polypeptide, or a protein having substantial or significant sequence identity or similarity to the reference polypeptide, and retains the biological activity of the reference polypeptide of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 100% identical, about 25% identical, about 30% identical, about 50% identical, about 65% identical, about 80% identical, about 90% identical, about 95% identical, or about 99% identical to the nucleic acid sequence encoding the parent CAR.

“Proliferative disease” as referred to herein means a unifying concept that excessive proliferation of cells and turnover of cellular matrix contribute significantly to the pathogenesis of several diseases, including cancer is presented.

“Administering” is referred to herein as providing the compositions of the invention to a patient. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration can be, for example, by bolus injection or by gradual perfusion over time. Alternatively, or concurrently, administration may be by the oral route. Additionally, administration may also be by surgical deposition of a bolus or pellet of cells, or positioning of a medical device.

Modified effector cell compositions described herein may comprises host cells expressing one or more nucleic acid sequences described herein, or a vector comprising one or more nucleic acid sequences described herein, in an amount that is effective to treat or prevent proliferative disorders. As used herein, the terms “treatment,” “treating,” and the like refer to obtaining a desired pharmacologic and/or physiologic effect. In embodiments, the effect is therapeutic, i.e., the effect partially or completely cures a disease and/or adverse symptom attributable to the disease. To this end, the inventive method comprises administering an “amount” of the composition comprising the host cells expressing the inventive nucleic acid sequence, or a vector comprising the inventive nucleic acid sequences.

An “amount” or “dose” refer to an amount effective, at dosages and for periods of time necessary, to achieve a desired therapeutic result. The amount may vary according to factors such as the disease state, age, sex, and weight of the individual, and the ability of the inventive nucleic acid sequences to elicit a desired response in the individual.

Alternatively, the pharmacologic and/or physiologic effect may be “prophylactic,” i.e., the effect completely or partially prevents a disease or symptom thereof.

A “prophylactically effective amount” refers to an amount effective, at dosages and for periods of time necessary, to achieve a desired prophylactic result (e.g., prevention of disease onset).

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Example 1. Manufacture of CD19-Specific CAR-T Cells

The SB transposon, CoOpCD19RCD28/pSBSO, expresses the human codon optimized (CoOp) 2^{nd} generation _{CoOp}CD19RCD28 CAR under EF-1/HTLV hybrid composite promoter (InvivoGen) comprised of Elongation Factor-1α (EF-1α) and 5′ untranslated region of the Human T-Cell Leukemia Virus (HTLV). The SB transposase, SB11, under the cytomegalovirus (CMV) promoter is expressed in cis from the DNA plasmid pCMV-SB11.

CD19-specific T cells can be generated from mononuclear cells (MNC) derived from PB or UCB using SB transposition to introduce the CAR followed by addition of aAPC to numerically expand the T cells in a CAR-dependent manner. Ten cuvettes (2×10⁷ MNC/cuvette) are electroporated for each recipient using 15 μg of DNA plasmid (CD19RCD28/pSBSO) coding for transposon (CAR) and 5 μg of DNA plasmid (pCMV-SB11) coding for transposase (SB11). The day of electroporation is defined as “Day 0” of Stimulation cycle #1. As controls for flow cytometry and culture conditions, autologous T cells are mock electroporated (without DNA plasmid) and numerically expanded on γ-irradiated aAPC (clone #4) that had been pre-loaded with OKT3 to cross-link CD3 to sustain T cell proliferation. The efficiency of electrotransfer and viability of the T cells the day after electroporation is routinely assessed. The expression of EGFP from control DNA plasmid (designated pmaxGFP) and CAR at this initial time point reflects protein expression from the integrated and episomal plasmid. Typically, EGFP expression is measured at 60% the day after electroporation and CAR expression at about 40% with T cell viability between 40%-50%. Recursive additions of γ-irradiated aAPC in the presence of soluble recombinant human IL-2 and IL-21 retrieve T cells stably expressing CAR (CD19RCD28). CD3^{neg}CD56⁺ NK cells may be depleted from the culture using CD56-specific paramagnetic beads if the percentage of these NK cells is ≥10% and especially if the percentage of CAR expressed on the T cells is low. This depletion prevents the rapid overgrowth of NK cells which interferes with the ability of activating and propagating cells (AaPC) to sustain the proliferation of CAR⁺ T cells. On occasion, depletion of NK cells from CAR⁺ T cells is undertaken during the last two stimulation cycles, but this introduces a loss of desired cells due to co-expression of CD56 on some CAR⁺ T cells. The T cells are grown in a functionally closed system using VueLife culture bags past Day 14. A subset of the genetically modified and propagated T cells are typically cryopreserved at Day 14 or Day 21 to 23 (end of Stimulation cycles #2 or #3) of co-culture on aAPC to serve as a source of archived material for future analyses and to be thawed if unanticipated problems subsequently occur during the manufacturing process. T cells can be harvested on or about Day 21 to Day 28, for instance, Day 23 of culture. Such T cells can express CAR and can be >80% viable.

### Example 2. In Vivo Expansion of CAR-T Cells

A 40 year old male was diagnosed with B-cell acute lymphoblastic leukemia (B-ALL). The patient exhibited karyotype 47 XY, and +X and did not exhibit any CNS involvement. First line therapy hyperCVAD was administered, leading to an initial complete remission. Patient relapsed and exhibited biomarkers CD19, CD20, CD22, CRLF2, karyotype 47 XY, +X, and inv 17. Multiple salvage therapies were given. POMP maintenance therapy was subsequently started. The patient further developed graft-versus-host disease GVHD and was treated with prednisone and tacrolimus. Additional radiation therapy (XRT), immunotherapy, hyperCAVD and O-EPOCH were further administered to the patient. Further, lymphodepletion was induced in the patient with cyclophosphamide at 500 mg/m² and fludarabine at 30 mg/m² for 3 days. Following lymphodepletion, 10⁶ CAR-T cells/kg were infused into the patient on day 0. On day 14, the patient developed GVHD. About 1 mg/kg of prednisone was administered starting day 14. On day 18 (or Day 48 since administration of CAR-T cells), GVHD was resolved and prednisone was reduced to about 0.5 mg/kg. White blood cell count (WBC) was 2.7 and absolute lymphocyte count (ALC) was 200.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein, or combinations of one or more of these embodiments or aspects described therein may be employed in practicing the invention. It is intended that the following claims define the scope of the invention and that methods and structures within the scope of these claims and their equivalents be covered thereby.

1. A method comprising administering to a subject in need thereof:
a) an effector cell; and
b) a steroid in an amount effective to induce and/or sustain expansion of a population of the effector cell in the subject.

2. (canceled)

3. The method of claim 1, wherein the effector cell is a chimeric antigen receptor (CAR) T cell.

4.-5. (canceled)

6. The method of claim 3, wherein the CAR binds at least one of CD19, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MUC-16, MAGE-A1, h5T4, PSMA, TAG-72, EGFRvIII, CD123 and/or VEGF-R2.

7.-11. (canceled)

12. The method of claim 1, wherein the effector cell is a T cell, a natural killer cell or a natural killer T cell, a modified T cell, an engineered T-cell receptor (TCR) T cell, a modified natural killer cell, or a modified natural killer T cell.

13.-15. (canceled)

16. The method of claim 1, wherein the steroid is administered simultaneously or sequentially with the effector cell.

17.-27. (canceled)

28. The method of claim 1, wherein a first amount of steroid is administered to the subject for a first period of time, and a second amount of steroid is administered to the subject for a second period of time.

29. (canceled)

30. The method of claim 28, wherein the first amount of steroid is an amount sufficient for treating a condition, wherein the condition is graft versus host disease (GVHD) or cytokine release syndrome (CRS).

31.-32. (canceled)

33. The method of claim 28, wherein upon recovery or improvement of the condition, said second amount of steroid is administered to the subject.

34.-50. (canceled)

51. The method of claim 1, wherein the steroid comprises at least one of fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone and stanozolol.

52. The method of claim 1, further comprising administering a cytokine.

53. (canceled)

54. The method of claim 52, wherein the cytokine is co-expressed with the effector cell.

55. (canceled)

56. The method of claim 52, wherein the cytokine comprises an interferon, an interleukin, a chemokine, a colony-stimulating factor, a tumor necrosis factor, IL-2, IL-7, IL-12, IL-15, IL-21, IFNγ, TNF-α variants, mbIL-15 and/or a fusion of IL-15 and IL-15Rα.

57.-88. (canceled)

89. A method of inducing T cell engraftment and/or expansion in a subject in need thereof, comprising:
a) contacting a T cells ex vivo with a vector encoding a chimeric receptor to generate a modified T cell;
b) administering to the subject an amount of the modified T cell; and
c) administering to the subject a steroid in an amount effective to induce and/or sustain expansion of a population of the modified T cells in the subject.

90. The method of claim 89, wherein the vector is a lentivirus vector, a retroviral vector, a Sleeping Beauty transposon or a non-viral vector.

91. The method of claim 89, wherein the vector is a Sleeping Beauty transposon.

92. The method of claim 89, wherein the modified T cell is a chimeric antigen receptor (CAR) T cell or an engineered T-cell receptor (TCR) T cell.

93.-97. (canceled)

98. The method of claim 89, wherein the chimeric receptor binds to at least one of CD19, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MAGE-A1, h5T4, PSMA, TAG-72, EGFRvIII, CD123 and/L VEGF-R2.

99.-163. (canceled)

164. The method of claim 89, wherein the chimeric receptor recognizes an epitope on CD19 to generate a CD19-specific T cell.

165.-170. (canceled)

171. A method of inducing T cell expansion in vivo, comprising:
contacting a population of modified T cells in vivo with a first amount of steroid effective to induce and/or sustain expansion of the population of the modified T cells, wherein said modified T cells comprise at least one chimeric receptor expressed on the cell surface.

172.-215. (canceled)

216. A system for inducing modified effector cell population expansion in vivo, comprising:
a population of modified effector cells, and an amount of at least one steroid,
wherein contacting a population of said modified effector cells in vivo with an effective amount of said at least one steroid, results in an expansion of the population of said modified effector cells.

217.-219. (canceled)
